(19) United States
(12) Patent Application Publication
(10) Pub. No.: US 2025/0082325 A1
(43) Pub. Date: Mar. 13, 2025

(54) END TOOL, CARTRIDGE, SURGICAL INSTRUMENT, AND OPERATING METHOD OF SURGICAL INSTRUMENT

(71) Applicant: LIVSMED INC., Seongnam-si (KR)

(72) Inventors: Jae Yeong LEE, Seongnam-si (KR); Hee Jin KIM, Seoul (KR); Jwa Nyun KIM, Yongin-si (KR); Dong Hoon KANG, Seoul (KR); Tae Jin PARK, Seongnam-si (KR)

(21) Appl. No.: 18/764,908

(22) Filed: Jul. 5, 2024

(30) Foreign Application Priority Data
Sep. 12, 2023 (KR) 10-2023-0121406

(51) Int. Cl. A61B 17/072; A61B 17/00; A61B 90/00

(52) U.S. Cl. CPC A61B 17/07207; A61B 17/00234; A61B 90/06; A61B 2017/00022; A61B 2017/00477; A61B 2017/07271; A61B 2090/064; A61B 2090/067

(57) ABSTRACT

An end tool of a surgical instrument includes a jaw and a sensing member, wherein the jaw is configured to accommodate at least one area of a working member that is movable in one direction, and the sensing member is configured to sense information about a position of the working member.

### CROSS-REFERENCE TO RELATED APPLICATION

This application is based on and claims priority under 35 U.S.C. § 119 to Korean Patent Application No. 10-2023-0121406, filed on Sep. 12, 2023, in the Korean Intellectual Property Office, the disclosure of which is incorporated by reference herein in its entirety.

### BACKGROUND

### 1. Field

The disclosure relates to an end tool, a cartridge, a surgical instrument, and an operating method of the surgical instrument.

### 2. Description of the Related Art

Recently, laparoscopic surgery, which is capable of reducing postoperative recovery time and complications through small incisions, has been actively used. Laparoscopic surgery is a method of performing surgery by drilling a plurality of small holes in a patient's abdomen and observing an abdominal cavity through the small holes. Laparoscopic surgery is widely used in general surgery.

In order to perform such laparoscopic surgery, various instruments are used. For example, a suture device that is inserted into a body is used to suture a surgical site within an abdominal cavity. A surgical stapler that sutures a surgical site by using medical staples is used as the suture device.

In general, a surgical stapler is a medical device that is often used for cutting and anastomosis of organs in abdominal and thoracic organ surgery. Such surgical staplers include an open stapler used in a thoracotomy or laparotomy state and an endo stapler used in thoracoscopy and laparoscopy.

Since surgical staplers are capable of simultaneously cutting a surgical site and performing anastomosis of organs, the surgical time is reduced and the surgical site is accurately sutured. In addition, surgical staplers are widely used in modern surgical procedures due to advantages of faster recovery and less scarring than when using surgical sutures for tissue cutting and suturing. In particular, surgical staplers are widely used in cancer surgery to cut cancer tissue and suture the cut site.

### SUMMARY

The disclosure provides an end tool, a cartridge, a surgical instrument, and an operating method of the surgical instrument, in which precise control of operation progress is facilitated when performing one or more operations included in laparoscopic surgery or various other surgeries.

Additional aspects will be set forth in part in the description which follows and, in part, will be apparent from the description, or may be learned by practice of the presented embodiments of the disclosure.

According to an embodiment, an end tool of a surgical instrument includes a jaw and a sensing member, wherein the jaw is configured to accommodate at least one area of a working member that is movable in one direction, and the sensing member is configured to sense information about a position of the working member.

In the present embodiment, the sensing member may be further configured to sense a distance to the working member.

In the present embodiment, the sensing member may be disposed closer to a proximal portion of the end tool than the working member.

In the present embodiment, the sensing member may be disposed closer to a distal portion of the end tool than the working member.

In the present embodiment, the sensing member may be disposed in an overlapped position when the working member moves and may be configured to sense the position of the working member.

In the present embodiment, the jaw may include a first jaw and a second jaw facing each other and disposed to move closer to or farther away from each other.

In the present embodiment, the first jaw and the second jaw of the jaw may be configured to rotate around one shaft so as to be closer to or farther away from each other.

In the present embodiment, the sensing member may be further configured to sense a distance between the first jaw and the second jaw.

In the present embodiment, the sensing member may be further configured to sense a load that the first jaw or the second jaw receives from a body tissue when the body tissue is located between the first jaw and the second jaw.

In the present embodiment, the sensing member may be further configured to sense a rotation angle when the first jaw and the second jaw are coupled to each other to perform a rotation motion.

In the present embodiment, the jaw may be further configured so that a staple is disposed thereon, and the staple may move through movement of the working member to perform stapling.

In the present embodiment, a wedge portion of the working member may push up the staple through the movement of the working member, and the stapling may be performed while a blade of the working member performs a cutting operation.

In the present embodiment, the jaw may be further configured to accommodate a cartridge, and the staple may be provided in plurality in the cartridge.

According to an embodiment, a cartridge applied to an end tool of a surgical instrument includes a housing, a plurality of staples disposed in the housing, a working member configured to be movable in at least one direction, and a sensing member configured to sense information about a position of the working member.

In the present embodiment, the sensing member may be further configured to sense a distance to the working member.

In the present embodiment, the sensing member may be disposed closer to a proximal portion of the cartridge than the working member.

In the present embodiment, the sensing member may be disposed closer to a distal portion of the cartridge than the working member.

In the present embodiment, the sensing member may be disposed in an overlapped position when the working member moves and may be configured to sense the position of the working member.

In the present embodiment, the working member may include a wedge portion having an inclined surface, and a blade formed on one side of the wedge portion and having an edge portion.

According to an embodiment, a surgical instrument includes the end tool, a manipulation portion configured to control an operation of the end tool, and a connection portion connecting the manipulation portion to the end tool.

In the present embodiment, the end tool may be configured to perform a yaw rotation motion around one axis and perform a pitch rotation motion around another axis.

According to an embodiment, an operating method of a surgical instrument including the end tool includes, in a surgical procedure including one or more operations using the surgical instrument, identifying the position of the working member through the sensing member before one operation is performed.

In the present embodiment, after identifying a standby state and a grip state of the jaw through the sensing member, stapling may be performed to release a staple through the movement of the working member and perform a tissue cutting process.

Other aspects, features, and advantages of the disclosure will become better understood through the accompanying drawings, the appended claims, and the detailed description.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic cross-sectional view of an end tool of a surgical instrument according to an embodiment;

FIGS. 2 and 3 are diagrams for describing a motion of a working member in the end tool of FIG. 1;

FIG. 4 is a schematic cross-sectional view of an end tool of a surgical instrument according to another embodiment;

FIGS. 5 and 6 are diagrams for describing a motion of a working member in the end tool of FIG. 4;

FIG. 7 is a schematic cross-sectional view of an end tool of a surgical instrument according to another embodiment;

FIGS. 8 and 9 are diagrams for describing a motion of a working member in the end tool of FIG. 7;

FIG. 10 is a schematic cross-sectional view of an end tool of a surgical instrument according to another embodiment;

FIGS. 11 and 12 are diagrams for describing a motion of a working member in the end tool of FIG. 10;

FIG. 13 is a schematic cross-sectional view of an end tool of a surgical instrument according to another embodiment;

FIG. 14 is a schematic cross-sectional view of an end tool of a surgical instrument according to another embodiment;

FIG. 15 is a schematic cross-sectional view of an end tool of a surgical instrument according to another embodiment;

FIGS. 16 and 17 are diagrams for describing a motion of a working member in the end tool of FIG. 15;

FIGS. 18 and 19 are diagrams illustrating a modification of the end tool of the surgical instrument of FIGS. 10 to 12;

FIG. 20 is a diagram illustrating an alternative embodiment of a working member of FIGS. 18 and 19;

FIGS. 21 and 22 are schematic cross-sectional views of an end tool of a surgical instrument according to another embodiment;

FIG. 23 is a schematic cross-sectional view of an end tool of a surgical instrument according to another embodiment;

FIG. 24 is a schematic cross-sectional view of an end tool of a surgical instrument according to another embodiment;

FIG. 25 is a schematic cross-sectional view of an end tool of a surgical instrument according to another embodiment;

FIG. 26 is a schematic cross-sectional view of an end tool of a surgical instrument according to another embodiment;

FIG. 27 is a schematic cross-sectional view of an end tool of a surgical instrument according to another embodiment;

FIG. 28 is an exploded perspective view illustrating a cartridge for an end tool of a surgical instrument according to an embodiment;

FIG. 29 is a combined perspective view illustrating the cartridge of FIG. 28;

FIG. 30 is a side view illustrating the cartridge of FIG. 28;

FIG. 31 is a perspective cross-sectional view illustrating the cartridge of FIG. 28;

FIG. 32 is a side cross-sectional view illustrating the cartridge of FIG. 28;

FIG. 33 is a perspective view illustrating a working member in the cartridge of FIG. 28;

FIGS. 34 and 35 are diagrams for describing a stapling operation of an end tool to which the cartridge of FIG. 28 is applied;

FIG. 36 is a cross-sectional view illustrating a cartridge for an end tool of a surgical instrument according to an embodiment;

FIG. 37 is a cross-sectional view illustrating a cartridge for an end tool of a surgical instrument according to another embodiment;

FIG. 38 is a cross-sectional view illustrating a cartridge for an end tool of a surgical instrument according to another embodiment;

FIG. 39 is a cross-sectional view illustrating a cartridge for an end tool of a surgical instrument according to another embodiment;

FIG. 40 is a schematic cross-sectional view of an end tool of a surgical instrument according to another embodiment;

FIGS. 41 and 42 are diagrams for describing a motion of a working member in the end tool of FIG. 40;

FIG. 43 is a perspective view for describing a working member of a cartridge in the end tool of FIG. 40;

FIG. 44 is a schematic perspective view of a surgical instrument according to an embodiment;

FIG. 45 is a side view of the surgical instrument of FIG. 44; and

FIG. 46 is a diagram for schematically describing an operation concept of a surgical instrument according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

As the present description allows for various changes and numerous embodiments, certain embodiments will be illustrated in the drawings and described in detail in the written description. Effects and features of the disclosure, and methods of achieving them will be clarified with reference to embodiments described below in detail with reference to the drawings. However, the disclosure is not limited to the following embodiments and may be embodied in various forms.

Hereinafter, embodiments will be described in detail with reference to the accompanying drawings. When describing embodiments with reference to the accompanying drawings, the same or corresponding elements are denoted by the same reference numerals, and redundant descriptions thereof are omitted.

It will be understood that although the terms “first,” “second,” etc. may be used herein to describe various elements, these elements should not be limited by these terms. These terms are only used to distinguish one element from another.

The singular forms as used herein are intended to include the plural forms as well unless the context clearly indicates otherwise.

It will be further understood that the terms “include” and/or “comprise” used herein specify the presence of stated features or elements, but do not preclude the presence or addition of one or more other features or elements.

Also, sizes of elements in the drawings may be exaggerated or reduced for convenience of explanation. For example, because sizes and thicknesses of elements in the drawings are arbitrarily illustrated for convenience of explanation, the disclosure is not limited thereto.

The x-axis, the y-axis, and the z-axis are not limited to three axes of the rectangular coordinate system and may be interpreted in a broader sense. For example, the x-axis, the y-axis, and the z-axis may be perpendicular to one another or may represent different directions that are not perpendicular to one another.

When a certain embodiment may be implemented differently, a specific process order may be performed differently from the described order. For example, two consecutively described processes may be performed substantially at the same time or performed in an order opposite to the stated order.

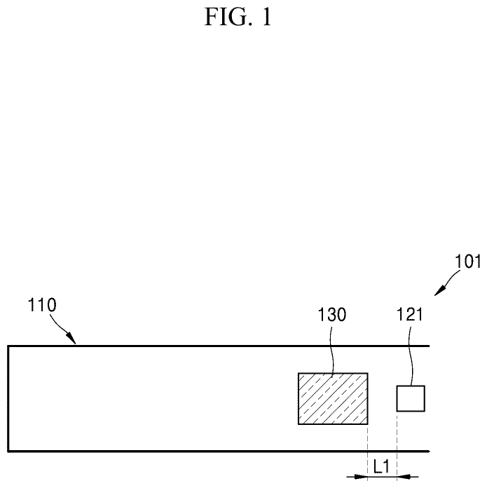

FIG. 1 is a schematic cross-sectional view of an end tool of a surgical instrument according to an embodiment.

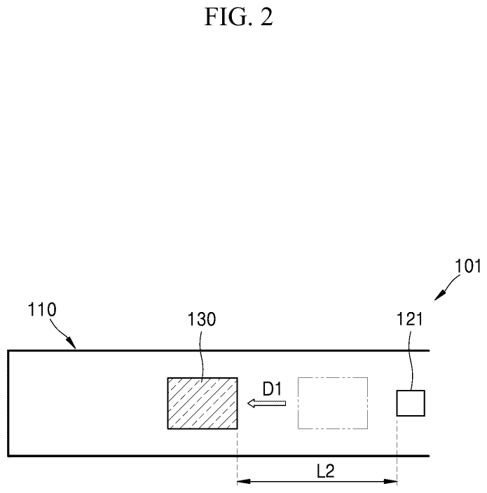

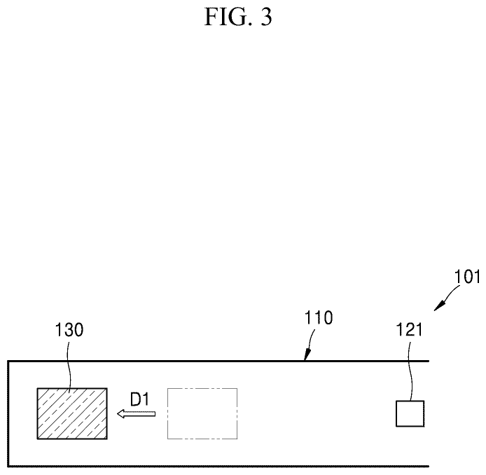

FIGS. 2 and 3 are diagrams for describing a motion of a working member in the end tool of FIG. 1.

Referring to FIG. 1, an end tool of a surgical instrument (hereinafter, an end tool 101) according to the present embodiment may include a jaw 110 and a sensing member 121.

The end tool 101 may be connected to an area of the surgical instrument, for example, an end portion of the surgical instrument, and inserted into a surgical site to perform operations required for surgery. In addition, as an example, the end tool 101 may be connected to a manipulation portion. As a specific example, the end tool 101 may be connected to the manipulation portion through one or more connection portions and may be driven through manipulation of the manipulation portion.

The jaw 110 may be configured to accommodate a working member 130 in at least one area. For example, the jaw 110 may be configured to accommodate the working member 130 in a space similar to a hollow box, and the working member 130 may move in one direction in a state of being accommodated in the jaw 110.

The jaw 110 may have various forms. For example, the jaw 110 may be provided with one or more jaws. As a specific example, the jaw 110 may be provided with two jaws. The working member 130 may be disposed in one of the jaws. In addition, the jaw 110 may have a space in which at least one staple (not shown) may be disposed.

As the working member 130 disposed in the jaw 110 moves, the end tool 101 may start one or more surgical operations. For example, as the working member 130 moves, the staple (not shown) accommodated in the jaw 110 may move so that stapling may be performed.

The sensing member 121 may be disposed in the jaw 110. As an example, the sensing member 121 may be disposed close to a proximal portion of the jaw 110. The proximal portion is an area including the end portion of the end tool 101 close to the connection portion that connects the end tool 101 to the manipulation portion. An area including an opposite end portion of the end tool 101 may be defined as a distal portion.

The sensing member 121 may be configured to identify information about the position of the working member 130.

For example, the sensing member 121 may include a distance detection sensor.

A distance L1 between the sensing member 121 and the working member 130 may be sensed through the sensing member 121, and the sensed distance or information about the position of the working member 130 through the distance may be transmitted in a form that may be identified by a user.

In addition, when the working member 130 moves in one direction D1 (for example, in the direction of the distal portion) as illustrated in FIG. 2, a changed distance L2 between the sensing member 121 and the working member 130 may be identified through the sensing member 121.

The distance to the working member 130 may be identified through the sensing member 121, and the information about the position of the working member 130 may be identified through the sensing member 121. Accordingly, when operation processes using the end tool 101 are performed, the state of each stage may be precisely identified and the start of progress of each stage may be precisely controlled.

Then, as illustrated in FIG. 3, the working member 130 may further move in one direction D1, for example, in the direction of the distal portion, so that the process using the end tool 101 may be completed. For example, stapling may be completed. As a result, each stage of the stapling process may be precisely controlled. For example, the start of each stage may be appropriately controlled. As a specific example, when the working member 130 moves in the direction of the distal portion while being in a position adjacent to the proximal portion, this may be precisely sensed and the process of waiting for or identifying whether to perform subsequent stages may be precisely controlled. As a specific example, when stapling is performed by using the jaw 110, a state after blood flows out from tissue on which the stapling is performed may be precisely controlled. Then, each stage may be safely and efficiently performed so that the working member 130 safely moves to complete stapling.

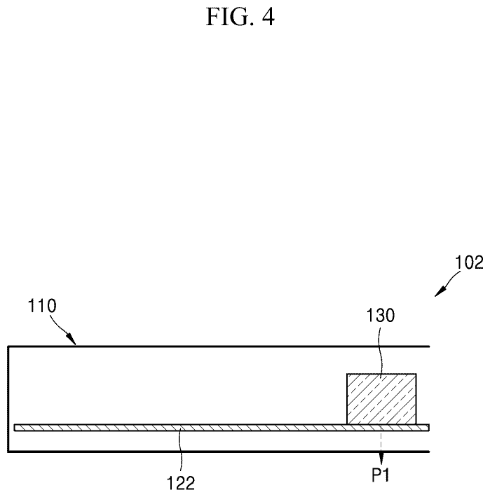

FIG. 4 is a schematic cross-sectional view of an end tool of a surgical instrument according to another embodiment.

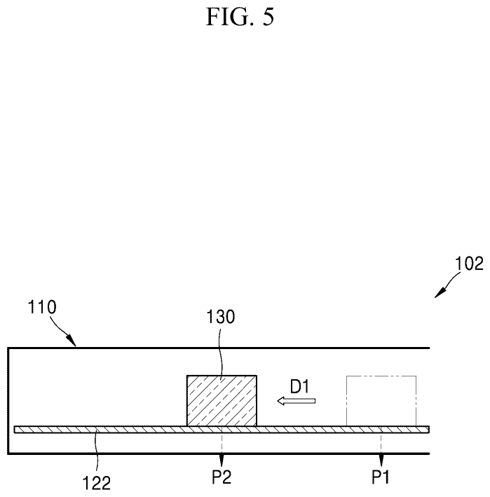

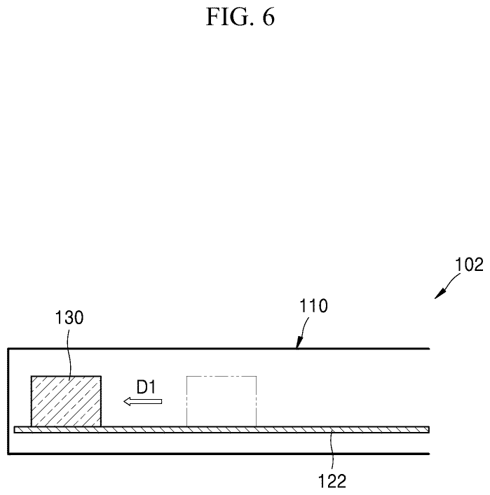

FIGS. 5 and 6 are diagrams for describing a motion of a working member in the end tool of FIG. 4.

Referring to FIG. 4, an end tool 102 according to the present embodiment may include a jaw 110 and a sensing member 122. For convenience of explanation, the following description focuses on the sensing member 122, which is the difference from the above-described embodiment.

The sensing member 122 may be disposed in the jaw 110 and may be configured to identify information about a position of a working member 130. For example, the sensing member 122 may include a position detection sensor. As a specific example, the sensing member 122 may be disposed to overlap the working member 130 on a movement path of the working member 130. For example, the sensing member 122 may have a shape that is longer than the working member 130 in the longitudinal direction of the jaw 110 on the movement path.

In an alternative embodiment, the sensing member 122 may include a capacitance-type sensor. The capacitance-type sensor may be used to easily sense the overlap or contact with the working member 130 when the working member 130 moves and to identify the information about the position of the working member 130. In another alternative embodiment, the sensing member 122 may include a pressure sensor. In addition, the sensing member 122 may include various other types of position sensors, and the shape or type of the sensing member 122 may be appropriately selected depending on the shape and size of the jaw 110.

A position P1 of the working member 130 may be identified through the sensing member 122. When the working member 130 moves in one direction D1, specifically in a direction of a distal portion, as illustrated in FIG. 5, a changed position P2 of the working member 130 may be identified through the sensing member 122.

By identifying information about the position of the working member 130 through the sensing member 122, the state of each stage when performing operations using the end tool 102 may be precisely identified and the start of progress of each stage may be precisely controlled.

Then, as illustrated in FIG. 6, the working member 130 may further move in one direction D1, specifically in the direction of the distal portion, so that the process using the end tool 102 is completed. For example, stapling may be completed. As a result, each stage of the stapling process may be precisely controlled. For example, the start of each stage may be appropriately controlled. As a specific example, when the working member 130 moves in the direction of the distal portion while being in a position adjacent to a proximal portion, this may be precisely sensed and the process of waiting for or identifying whether to perform subsequent stages may be precisely controlled.

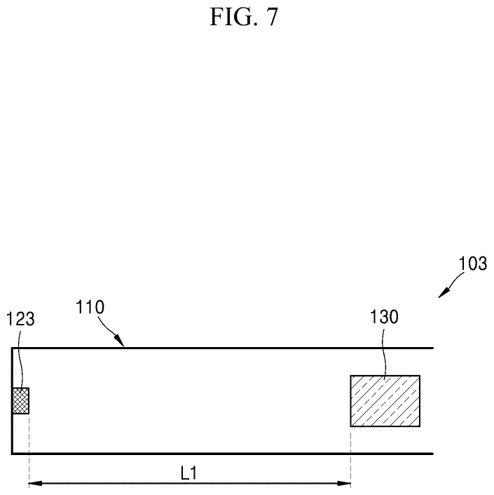

FIG. 7 is a schematic cross-sectional view of an end tool of a surgical instrument according to another embodiment.

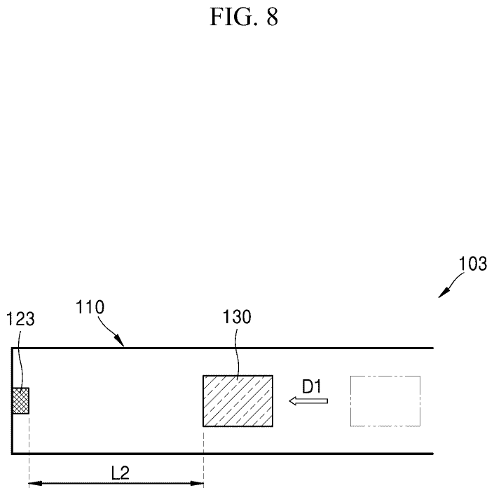

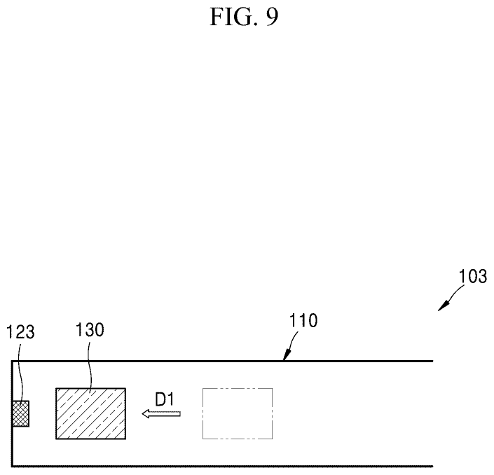

FIGS. 8 and 9 are diagrams for describing a motion of a working member in the end tool of FIG. 7.

Referring to FIG. 7, an end tool 103 according to the present embodiment may include a jaw 110 and a sensing member 123. For convenience of explanation, the following description focuses on the sensing member 123, which is the difference from the above-described embodiment.

The sensing member 123 may be configured to identify information about a position of a working member 130. For example, the sensing member 123 may include a distance detection sensor. The sensing member 123 may be disposed in the jaw 110. As an example, the sensing member 123 may be disposed close to a distal portion of the jaw 110. As a specific example, the sensing member 123 may be disposed closer to the distal portion of the jaw 110 than the working member 130.

A distance L1 between the sensing member 123 and the working member 130 may be sensed through the sensing member 123, and the sensed distance or information about the position of the working member 130 through the distance may be transmitted in a form that may be identified by a user.

When the working member 130 moves in one direction D1, specifically the indirection of the distal portion, as illustrated in FIG. 8, a changed distance L2 between the sensing member 123 and the working member 130 may be identified through the sensing member 123.

The information about the position of the working member 130 may be identified through the sensing member 123. In this manner, the state of each stage when performing operations using the end tool 103 may be identified and the start of each stage may be precisely controlled.

Then, as illustrated in FIG. 9, the working member 130 may further move in one direction D1, specifically in the direction of the distal portion, so that the process using the end tool 103 is completed. For example, stapling may be completed. As a result, each stage of the stapling process may be precisely controlled. For example, the start of each stage may be appropriately controlled. As a specific example, when the working member 130 moves in the direction of the distal portion while being in a position adjacent to a proximal portion, this may be precisely sensed and the process of waiting for or identifying whether to perform subsequent stages may be precisely controlled.

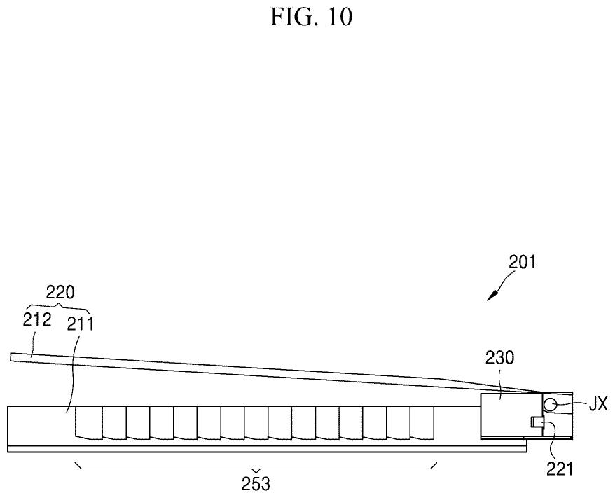

FIG. 10 is a schematic cross-sectional view of an end tool of a surgical instrument according to another embodiment.

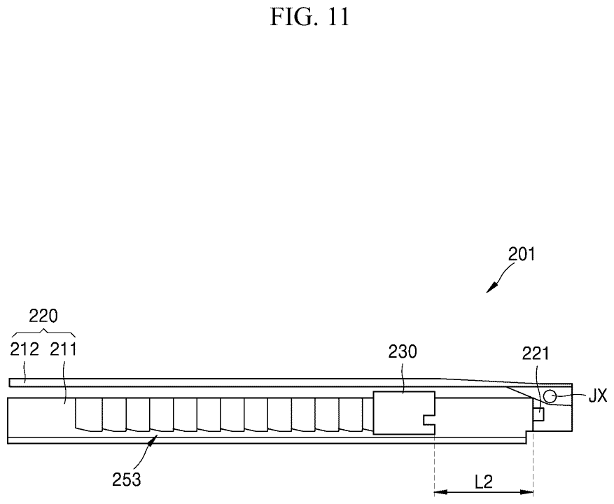

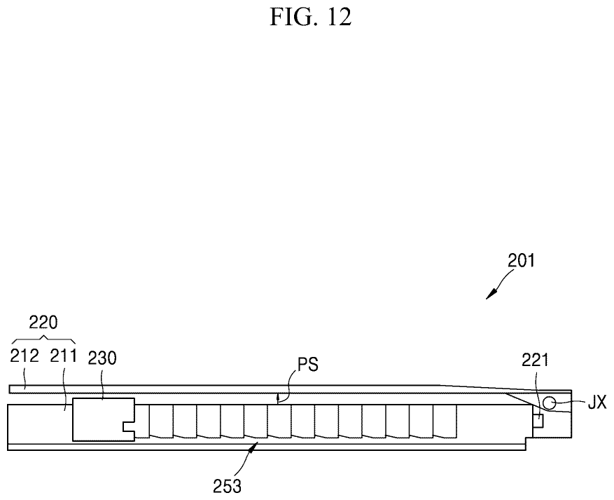

FIGS. 11 and 12 are diagrams for describing a motion of a working member in the end tool of FIG. 10.

Referring to FIG. 10, an end tool 201 of a surgical instrument according to the present embodiment may include a jaw 220 and a sensing member 221.

The end tool 201 may be connected to an area of the surgical instrument, for example, an end portion of the surgical instrument, and inserted into a surgical site to perform operations required for surgery. In addition, as an example, the end tool 201 may be connected to a manipulation portion. As a specific example, the end tool 201 may be connected to the manipulation portion through one or more connection portions and may be driven through manipulation of the manipulation portion.

The jaw 220 may be configured to include one or more elements. For example, the jaw 220 may include a first jaw 211 and a second jaw 212.

The first jaw 211 and the second jaw 212 may be disposed to face each other and may perform a grip operation. As a specific example, the first jaw 211 and the second jaw 212 may perform a grip operation while rotating around a rotational shaft JX.

The first jaw 211 may have an elongated shape in which a length thereof is greater than a width thereof. For example, the first jaw 211 may have an elongated rod shape. The first jaw 211 may be configured to accommodate a working member 230. For example, the first jaw 211 may be similar to a hollow box. The first jaw 211 may have one surface (e.g., the surface facing the second jaw 212) open. The first jaw 211 may be configured to accommodate the working member 230 therein. The first jaw 211 may move in one direction while the working member 230 is accommodated in the first jaw 211.

The second jaw 212 may have an elongated shape in which a length thereof is greater than a width thereof. For example, the second jaw 212 may have an elongated rod shape.

The first jaw 211 may have a space in which at least one staple 253 is disposed and may be formed so that the staple 253 is disposed in the interior similar to the hollow box described above.

In an alternative embodiment, an anvil may be formed on one surface of the second jaw 212 to face the staple 253.

The end tool 201 may start one or more operations through the movement of the working member 230 disposed in the first jaw 211. For example, due to the movement of the working member 230, the staple 253 accommodated in the first jaw 211 may be released toward the second jaw 212 to perform stapling.

The sensing member 221 may be disposed in the jaw 220. As an example, the sensing member 221 may be disposed in one area of the first jaw 211. As a specific example, the sensing member 221 may be disposed closer to a proximal portion of the first jaw 211 than the staple 253. The proximal portion refers to an area close to the rotational shaft JX of the jaw 220, and the distal portion refers to an area opposite to the proximal portion.

The sensing member 221 may be configured to identify information about the position of the working member 230.

For example, the sensing member 221 may include a distance detection sensor.

The distance between the sensing member 221 and the working member 230 may be sensed through the sensing member 221, and the sensed distance or information about the position of the working member 230 through the distance may be transmitted in a form that may be identified by a user.

In addition, when the working member 230 moves in the direction of the distal portion as illustrated in FIG. 11, a changed distance L2 between the sensing member 221 and the working member 230 may be identified through the sensing member 221.

The distance to the working member 230 may be identified through the sensing member 221, and the information about the position of the working member 230 may be identified through the sensing member 221. In this manner, the state of each stage when performing operations using the end tool 201 may be precisely identified, the start of the each stage may be precisely controlled, and the end of the operation may be precisely controlled as necessary.

For example, a state in which the first jaw 211 and the second jaw 212 are closed and then disposed immediately before the working member 230 pushes up the staple 253 (for example, FIG. 11) may be easily identified. By identifying such a state, a waiting time for blood to flow out from tissue between the first jaw 211 and the second jaw 212 due to the grip of the first jaw 211 and the second jaw 212 may be easily maintained.

Then, as illustrated in FIG. 12, the working member 230 may move forward in the direction of the distal portion and the staple 253 may be released in the release direction DS to perform stapling. As a result, each stage of the stapling process may be precisely controlled. For example, the start of each stage may be appropriately controlled. As a specific example, after the working member 230 moves forward in a state of being in a position adjacent to the proximal portion (a standby state) and the grip state of the first jaw 211 and the second jaw 212 is easily identified through the sensing member 221, a state (a grip completion state) after blood flows out from tissue between the first jaw 211 and the second jaw 212 may be precisely controlled. Then, the working member 230 may move safely and a stapling state (a firing state) may be performed effectively.

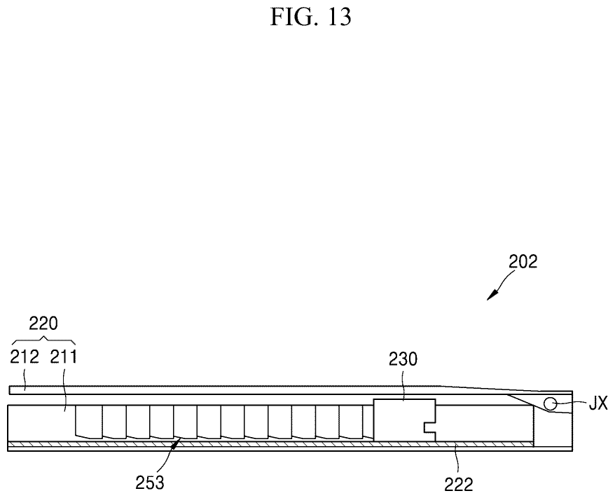

FIG. 13 is a schematic cross-sectional view of an end tool of a surgical instrument according to another embodiment.

Referring to FIG. 13, an end tool 202 according to the present embodiment may include a jaw 220 and a sensing member 222. The jaw 220 may be configured to include one or more elements. For example, the jaw 220 may include a first jaw 211 and a second jaw 212. Since the jaw 220 is the same as the jaw 220 of FIGS. 10 to 12 according to the above-described embodiment or may be modified and applied within a similar range as necessary, a detailed description thereof is omitted.

The sensing member 222 may be disposed in the jaw 220. As an example, the sensing member 222 may be disposed in one area of the first jaw 211.

The sensing member 222 may be configured to identify information about a position of a working member 230. For example, the sensing member 222 may include a position detection sensor. As a specific example, the sensing member 222 may be disposed to overlap the working member 230 on a movement path of the working member 230. As an example, the sensing member 222 may have a shape that is longer than the working member 230 in the longitudinal direction of the first jaw 211 on the movement path.

In addition, the sensing member 222 may be disposed below the first jaw 211. For example, the sensing member 222 be disposed farther away from the second jaw 212 than the working member 230. As a specific example, the sensing member 222 may be disposed closer to the inner bottom surface of the first jaw 211 than a staple 253 so that the working member 230 is located between the sensing member 222 and the second jaw 212.

In an alternative embodiment, the sensing member 222 may include a capacitance-type sensor. The capacitance-type sensor may be used to easily sense the overlap or contact with the working member 130 when the working member 130 moves and to identify the information about the position of the working member 130. In another alternative embodiment, the sensing member 222 may include a pressure sensor. In addition, the sensing member 222 may include various other types of position sensors, and the shape or type of the sensing member 222 may be appropriately selected depending on the shape and size of the first jaw 211.

The information about the position of the working member 230 through the sensing member 222 may be transmitted in a form that may be identified by a user.

The position that changes when the working member 230 moves may be identified through the sensing member 222. In this manner, the state of each stage when performing operations using the end tool 202 may be precisely identified, the start of progress of each stage may be precisely controlled, and the end of the operation may be precisely controlled as necessary.

For example, a waiting time for blood to flow out from tissue between the first jaw 211 and the second jaw 212 due to the grip of the first jaw 211 and the second jaw 212 may be easily maintained, and a process in which the staple 253 is released in the release direction DS to perform stapling may be precisely controlled.

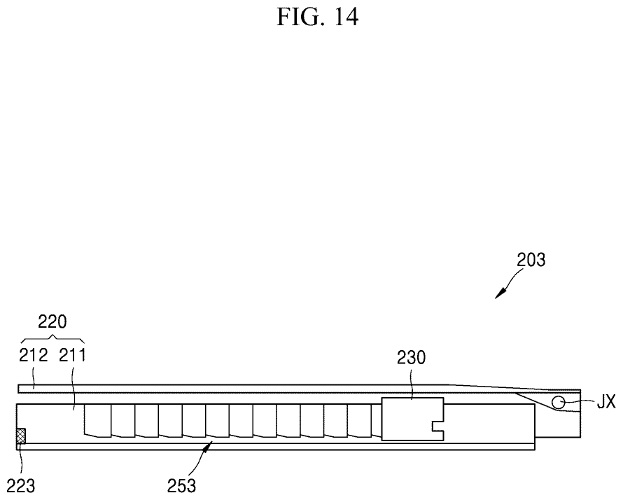

FIG. 14 is a schematic cross-sectional view of an end tool of a surgical instrument according to another embodiment.

Referring to FIG. 14, an end tool 203 according to the present embodiment may include a jaw 220 and a sensing member 223. The jaw 220 may be configured to include one or more elements. For example, the jaw 220 may include a first jaw 211 and a second jaw 212. Since the jaw 220 is the same as the jaw 220 of FIGS. 10 to FIG. 12 according to the above-described embodiment or may be modified and applied within a similar range as necessary, a detailed description thereof is omitted.

The sensing member 223 may be disposed in the jaw 220. As an example, the sensing member 223 may be disposed in one area of the first jaw 211. As a specific example, the sensing member 223 may be disposed closer to a distal portion of the first jaw 211 than a staple 253.

The sensing member 223 may be configured to identify information about a position of a working member 230. For example, the sensing member 223 may include a distance detection sensor.

In this manner, the distance between the sensing member 223 and the working member 230 may be sensed, and the sensed distance or information about the position of the working member 230 through the distance may be transmitted in a form that may be identified by a user.

The information about the position of the working member 230 may be identified through the sensing member 223, the state of each stage when performing operations using the end tool 203 may be precisely identified, the start of progress of each stage may be precisely controlled, and the end of the operation may be precisely controlled as necessary.

For example, a waiting time for blood to flow out from tissue between the first jaw 211 and the second jaw 212 due to the grip of the first jaw 211 and the second jaw 212 may be easily maintained, and a process in which a staple 253 is released in a release direction DS to perform stapling may be precisely controlled.

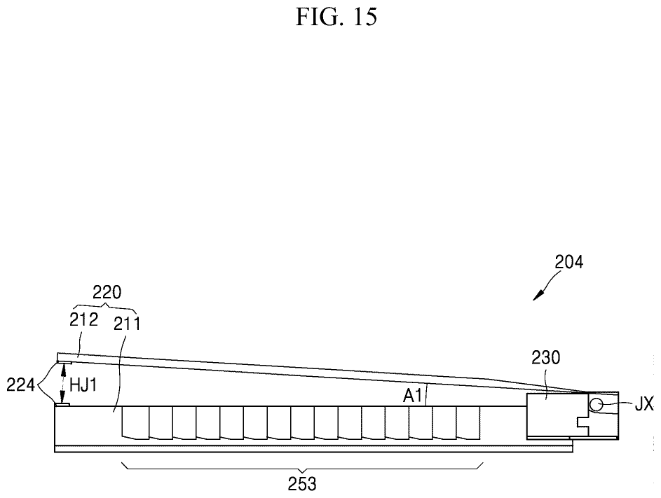

FIG. 15 is a schematic cross-sectional view of an end tool of a surgical instrument according to another embodiment.

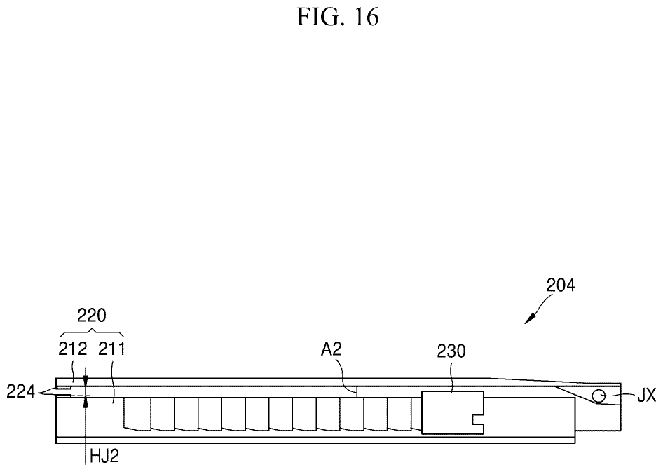

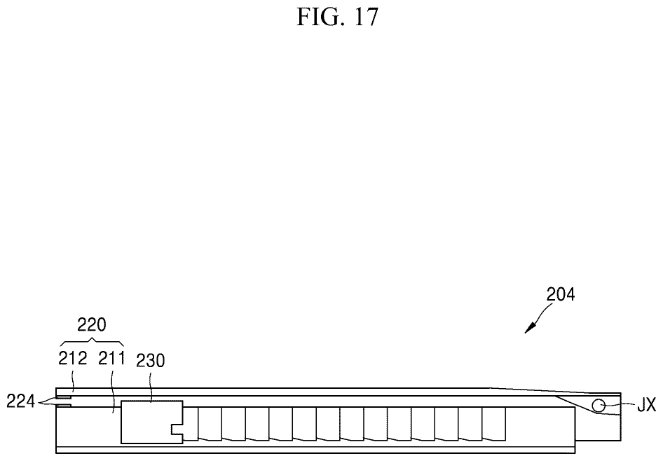

FIGS. 16 and 17 are diagrams for describing a motion of a working member in the end tool of FIG. 15.

Referring to FIG. 15, an end tool 204 according to the present embodiment may include a jaw 220 and a sensing member 224. The jaw 220 may be configured to include one or more elements. For example, the jaw 220 may include a first jaw 211 and a second jaw 212. Since the jaw 220 is the same as the jaw 220 of FIGS. 10 to FIG. 12 according to the above-described embodiment or may be modified and applied within a similar range as necessary, a detailed description thereof is omitted.

The sensing member 224 may be configured to sense a value of a gap between the first jaw 211 and the second jaw 212 of the jaw 220, for example, a gap between ends of distal portions or areas adjacent thereto.

As a specific example, the sensing member 224 may be disposed in each of an area adjacent to a distal portion of the first jaw 211 and facing the second jaw 212 and an opposite area adjacent to a distal portion of the second jaw 212 and facing the first jaw 211. Alternatively, the sensing member 224 may be formed in either the first jaw 211 or the second jaw 212.

The sensing member 224 may include a position detection sensor or a distance detection sensor. The sensing member 224 may sense a gap HJ1 between the first jaw 211 and the second jaw 212, and the sensed distance or information about a position of a working member 230 through the distance may be transmitted in a form that may be identified by a user. For example, a state (a standby state) in which the working member 230 is in a position adjacent to a proximal portion may be identified. This may be transmitted as information that may be identified by the user.

In addition, when the working member 230 moves in the direction of the distal portion as illustrated in FIG. 16, a reduced gap HJ2 between the first jaw 211 and the second jaw 212 may be sensed through the sensing member 224. In this manner, it may be identified that the working member 230 moves forward and the first jaw 211 and the second jaw 212 are in a grip state.

After identifying the grip state and then passing a grip completion state through stable standby, the working member 230 may further move forward to an area close to the distal portion as illustrated in FIG. 17.

In an alternative embodiment, the end tool 204 may include a sensing member (not shown) that senses an angle A1 between the first jaw 211 and the second jaw 212 in FIG. 15 and a changed angle A2 in FIG. 16. Accordingly, the gap between the first jaw 211 and the second jaw 212 may be indirectly identified, instead of the sensing members 224 disposed in each of the first jaw 211 and the second jaw 212. The sensing member that senses the angle A1 may have various shapes. For example, the sensing member may include an optical angle measuring member and a contact-type angle measuring member. As another example, the sensing member may include a rotation measuring member that measures the amount of rotation when the first jaw 211 and the second jaw 212 rotate around the rotational shaft JX.

The state of each stage when performing operations using the end tool 204 may be precisely identified through the sensing member 224 or the sensing member (not shown) that senses the angle A1, the start of progress of each stage may be precisely controlled, and the end of the operation may be precisely controlled as necessary.

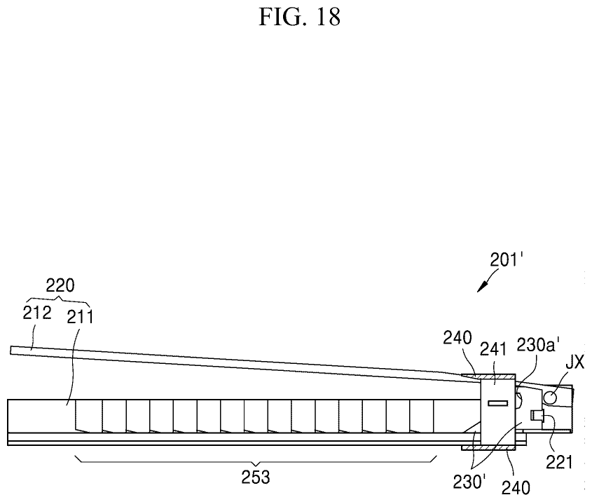

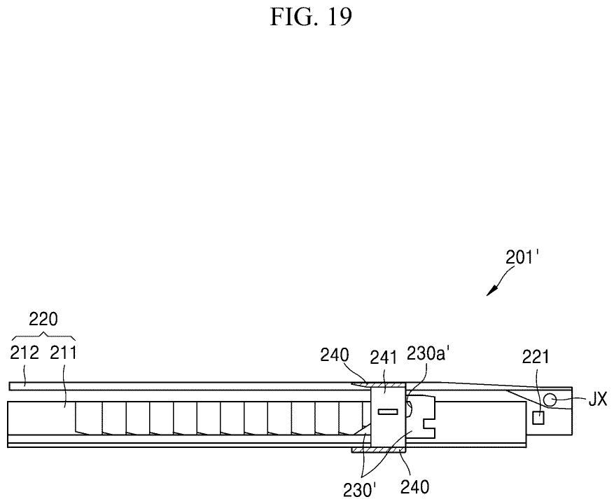

FIGS. 18 and 19 are diagrams illustrating a modification of the end tool of the surgical instrument of FIGS. 10 to 12. As a specific example, a working member 230′ and a working guide portion 240 may be modified or added.

Although not illustrated for convenience of explanation, FIGS. 18 and 19 may also be applied as modifications of the end tools 202, 203, and 204 of FIGS. 13 to 17.

Referring to FIGS. 18 and 19, an end tool 201′ according to the present embodiment may include a jaw 220 and a sensing member 221. The jaw 220 may include a first jaw 211 and a second jaw 212. Since the jaw 220 is the same as the jaw 220 of FIGS. 10 to 12 according to the above-described embodiment or may be modified and applied within a similar range as necessary, a detailed description thereof is omitted.

For example, the sensing member 221 may include a distance detection sensor. The distance between the sensing member 221 and a working member 230′ may be sensed through the sensing member 221. Since the sensing member 221 is the same as the sensing member 221 illustrated in FIGS. 10 to 12 or may be modified and applied within a similar range as necessary a detailed description thereof is omitted.

In addition, when the working member 230′ moves in a direction of a distal portion as illustrated in FIG. 19, a changed distance L2 between the sensing member 221 and the working member 230′ may be identified through the sensing member 221.

The working member 230′ may be configured to push up a staple 253 and may be configured to include, for example, a wedge having an inclined surface.

In addition, the working member 230′ may include a blade 230a′. A stapled tissue may be cut through the blade 230a′.

A working guide portion 240 may be further disposed to close the first jaw 211 and the second jaw 212. For example, the working guide portion 240 may have at least two members to support the first jaw 211 and the second jaw 212 on both sides and may include a side member 241 connecting the first jaw 211 and the second jaw 212 to each other.

After the first jaw 211 and the second jaw 212 are maintained in a closed state through the working guide portion 240, stapling may be performed through the working member 230′, and thus, the grip state described above may be performed more stably.

In an alternative embodiment, the working member 230′ and the working guide portion 240 may be connected to each other so that the working member 230′ and the working guide portion 240 may move together.

The closed state of the first jaw 211 and the second jaw 212 and the gap therebetween may be controlled through the working guide portion 240 as much as desired, for example, as much as the gap between the working guide portions 240 on both sides. Accordingly, the thickness of the tissue disposed between the first jaw 211 and the second jaw 212 may be compressed to easily ensure stable performance of stapling and incision.

For example, the compression force of the tissue and the uniformity of compression may be improved by applying higher compression force through the working guide portion 240 rather than applying compressive force to the desired tissue between the first jaw 211 and the second jaw 212 with only the grip force of the first jaw 211 and the second jaw 212.

In addition, when the working guide portion 240 on both sides are disposed above and below the working member 230′, the widening may be reduced or prevented due to tissue elasticity or the like by controlling the gap between the first jaw 211 and the second jaw 212 near the working member 230′. Accordingly, compressing the tissue to the intended thickness may be performed efficiently.

In the standby state of FIG. 18, the working guide portion 240 does not correspond to the first jaw 211 and the second jaw 212, and thus, the first jaw 211 and the second jaw 212 may maintain an open state. As illustrated in FIG. 19, the working member 230′ and the working guide portion 240 may move forward in the direction of the distal portion. The first jaw 211 and the second jaw 212 may maintain the grip state stably through the working guide portion 240. After such a state is easily confirmed through the sensing member 221, the state (the grip completion state) after blood flows out from the tissue may be precisely controlled. Then, the working member 230′ may move safely and the stapling state (the firing state) may be performed effectively.

Although not illustrated, the configurations of the working member 230′ and the working guide portion 240 of FIGS. 18 and 19 may be applied to the end tools 202, 203, and 204 of FIGS. 13 to 17.

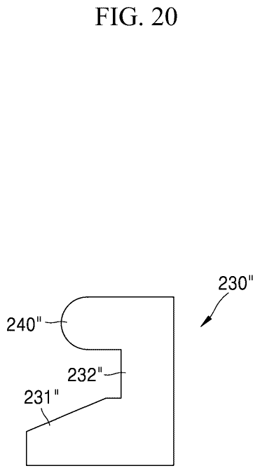

FIG. 20 is a diagram illustrating an alternative embodiment of the working member of FIGS. 18 and 19.

Referring to FIG. 20, a working member 230″ may include a wedge portion 231″, a blade 232″, and a bent portion 240″.

The wedge portion 231″ may push up the staple and the blade 232″ may cut the stapled tissue. The bent portion 240″ may be formed on one side of the blade 232″, for example, in an area opposite to an area adjacent to the wedge portion 231″. Accordingly, the blade 232″ may be disposed between the wedge portion 231″ and the bent portion 240″.

At least one area of the bent portion 240″ may be disposed in the second jaw 212, the first jaw 211 and the second jaw 212 may be closed effectively, and a load applied to the wedge portion 231″ during stapling may be dispersed. Accordingly, the stability and control ability of the motion of the working member 230″ during stapling may be improved.

On the other hand, unlike the working guide portion 240 described above, the bent portion 240″ according to the present embodiment may be disposed on the second jaw 212, instead of being disposed on both sides of the jaw. In this manner, precise stapling control may be implemented by reducing an occurrence of tilt moment in the forward and backward direction, which is caused by frictional force occurring on both sides of the first jaw 211 and the second jaw 212 when the working member 230″ moves forward. In order to increase such an effect, the bent portion 240″ may be variously designed according to the shapes of the first jaw 211 and the second jaw 212. As an example, an area that is in contact with the second jaw 212 may include a curved area.

In addition, the bent portion 240″ may be transformed into various forms depending on the position where the bent portion 240″ is disposed, the shape, size, and material of the first jaw 211 and the second jaw 212, or the friction therebetween.

Although not illustrated, in an alternative embodiment, a groove in which at least one area of the bent portion 240″ may be disposed may be further formed in the second jaw 212.

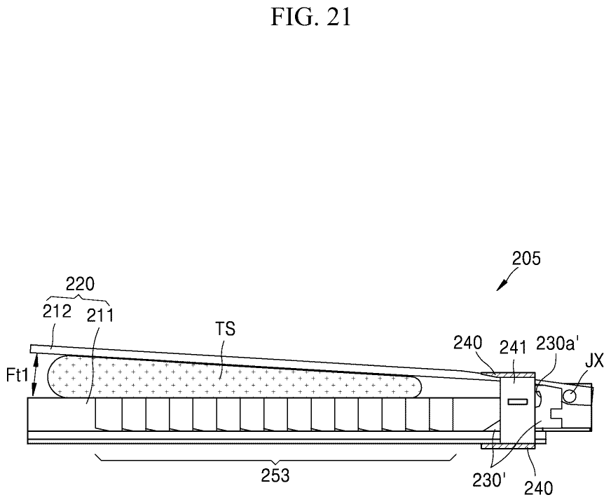

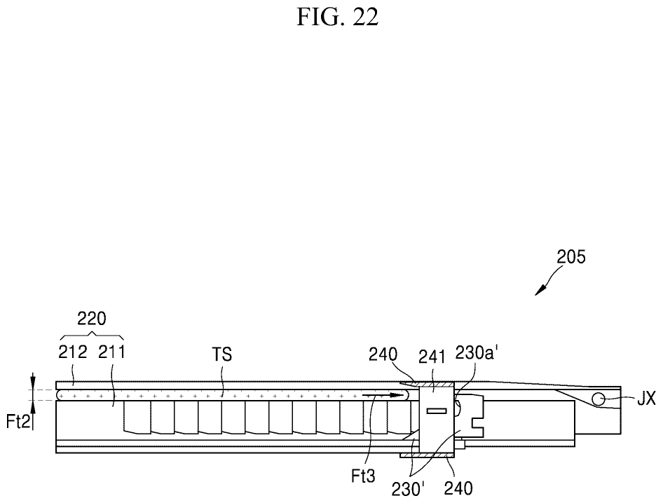

FIGS. 21 and 22 are schematic cross-sectional views of an end tool of a surgical instrument according to another embodiment.

Referring to FIG. 21, an end tool 205 according to the present embodiment may include a jaw 220 and a sensing member (not shown). The sensing member may include a load sensing member that measures a load. The jaw 220 may be configured to include one or more elements. For example, the jaw 220 may include a first jaw 211 and a second jaw 212. Since the jaw 220 is the same as the jaw 220 of FIGS. 10 to FIG. 12 according to the above-described embodiment or may be modified and applied within a similar range as necessary, a detailed description thereof is omitted.

The end tool 205 according to the present embodiment may sense loads received by the first jaw 211 and the second jaw 212 of the jaw 220. For example, when tissue TS is located between the first jaw 211 and the second jaw 212 as illustrated in FIG. 21, a load Ft1 that the first jaw 211 or the second jaw 212 receives due to the tissue TS may be sensed.

For example, a pressure sensor and the like may be disposed in one area of the first jaw 211 and the second jaw 212. In addition, a rotation resistance measuring unit that measures resistance in the rotational direction of the first jaw 211 and the second jaw 212 in an area where the first jaw 211 and the second jaw 212 are connected to each other, as a specific example, in an area adjacent to a rotational shaft JX.

In addition, in an alternative embodiment, a working guide portion 240 may be further disposed to close the first jaw 211 and the second jaw 212. For example, the working guide portion 240 may have at least two members to support the first jaw 211 and the second jaw 212 on both sides and may include a side member 241 that connects the first jaw 211 and the second jaw 212 to each other. At this time, the load received by at least one of the first jaw 211 and the second jaw 212 through the two members on both sides of the working guide portion 240, for example, the load received in a direction away from each other may be measured.

Through the measurement of the load between the first jaw 211 and the second jaw 212, it may be identified that a state in which the tissue TS is disposed between the first jaw 211 and the second jaw 212, that is, a state (a standby state) in which the working member 230 is in a position adjacent to the proximal portion is maintained without sufficiently closing the first jaw 211 and the second jaw 212. This may be transmitted as information that is recognizable by the user.

In addition, as illustrated in FIG. 22, when the working member 230 moves in the direction of the distal portion and the first jaw 211 and the second jaw 212 are closed with the tissue TS therebetween, and thus, a load Ft2 therebetween increases, the change in load may be sensed through the sensing member. In this manner, for example, it may be confirmed that the working member 230 is moving forward and the first jaw 211 and the second jaw 212 are in a grip state.

After identifying the grip state and passing the grip completion state through a stable standby, the working member 230 may further move forward to an area close to the distal portion. Accordingly, stapling may be performed stably.

when operations using the end tool 205 are performed through load sensing between the first jaw 211 and the second jaw 212, the state of each stage may be precisely identified, the start of progress of each stage may be precisely controlled, and the end of the operation may be precisely controlled as necessary.

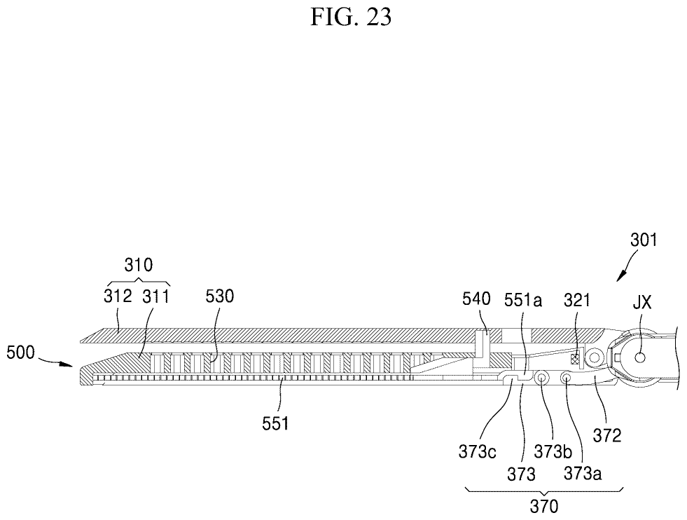

FIG. 23 is a schematic cross-sectional view of an end tool of a surgical instrument according to another embodiment.

Referring to FIG. 23, an end tool 301 of a surgical instrument according to the present embodiment may include a jaw 310 and a sensing member 321.

The sensing member 321 may be disposed in the jaw 310. As an example, the sensing member 321 may be disposed in one area of a first jaw 311. As a specific example, the sensing member 321 may be disposed closer to a proximal portion of the first jaw 311. The sensing member 321 may be configured to identify information about a position of a working member 540, and a detailed description thereof may be the same as described in the embodiment of FIGS. 10 to 12.

The jaw 310 according to the present embodiment is described in more detail.

The first jaw 311 may be formed in an elongated bar shape as a whole. In a distal portion, the first jaw 311 may be configured to accommodate a cartridge 500. In the proximal portion, a power transmission member (for example, a pulley) may be coupled to the first jaw 311. The first jaw 311 may be configured to be rotatable around a rotational shaft JX. As an example, the first jaw 311 may be formed in a shape of a generally hollow box with one surface (an upper surface) removed. A cartridge accommodation space capable of accommodating the cartridge 500 may be formed inside the first jaw 311. As a specific example, the first jaw 311 may have a cross-section of approximately a ‘U’ shape.

A guide groove (not shown) may be formed on one side of the first jaw 311, for example, in the proximal portion, to guide movement of a staple link assembly 370 to be described below. The guide groove (not shown) may be formed in a shape of a groove formed along a movement path of the staple link assembly 370. As a first protrusion 373a and a second protrusion 373b of a second link 373 of the staple link assembly 370 formed in a protrusion shape move along the guide groove while the first protrusion 373a and the second protrusion 373b are fitted into the groove-shaped guide groove (not shown), the staple link assembly 370 may move relative to the first jaw 311 (and a cartridge 500 therein).

The second jaw 312 may be formed in an elongated bar shape as a whole. As an example, an anvil may be formed on the distal portion side, a power transmission member (for example, a pulley) may be coupled to the proximal portion, and the second jaw 312 may be configured to be rotatable around the rotational shaft JX.

As an example, the anvil (not shown) of the second jaw 312 may be formed in a flat plane shape. Shapes corresponding to the shape of the staple 530 may be formed on one surface of the anvil of the second jaw 312. The anvil may serve as a support for supporting the opposite side of the working member 540 when the working member 540 pushes up the staple 530 during the stapling operation, so that the staple 530 is bent.

The working member 540 may be disposed inside the cartridge 500. As another example, the working member 540 may be disposed in the first jaw 311, separately from the cartridge 500, and then, may be coupled to the cartridge 500 when the cartridge 500 is accommodated in the first jaw 311.

The working member 540 may have various shapes and may include, for example, a wedge and a blade.

On the other hand, the end tool 301 according to the present embodiment may further include the staple link assembly 370. The staple link assembly 370 may include one or more link members 372 and 373. The staple link assembly 370 may be connected to a reciprocating member 551 of a reciprocating assembly of the cartridge 500 and may transmit driving force for movement of the working member 540.

In an alternative embodiment, the link members 372 and 373 may include a first link 372 and a second link 373 and may have various forms that transmit force. For example, the first link 372 may be formed in an elongated bar shape. Through-holes may be formed at both end portions of the first link 372. The second link 373 may be inserted into the through-hole formed at one end portion of the first link 372. The second link 373 may be formed in an elongated bar shape and may be coupled to the first link 372. As an example, the second link 373 may include a first protrusion 373a, a second protrusion 373b, and a fastening portion 373c.

The first protrusion 373a of the second link 373 may be fitted into the through-hole of the first link 372 and axially coupled thereto so that the second link 373 may be coupled to the first link 372. In addition, the first protrusion 373a may be fitted into the guide groove (not shown) of the first jaw 311 as described above, and the second protrusion 373b in an area of the central portion of the second link 373 may also be fitted into the guide groove (not shown) of the first jaw 311.

As such, the first protrusion 373a and the second protrusion 373b of the second link 373, which are formed in the protrusion shape, may move in a state of being fitted into the groove-shaped guide groove, and thus, the staple link assembly 370 may move relative to the first jaw 311 (and the cartridge 500 therein). For example, the staple link assembly 370 may move linearly by receiving driving force through the rotation of the power transmission member (as a specific example, the pulley (not shown)) coupled to one area of the staple link assembly 370. Due to such a motion, the working member 540 may move.

The distance to the working member 540 may be identified through the sensing member 321, and the information about the position of the working member 540 may be identified through the sensing member 221. In this manner, the state of each stage when performing operations using the end tool 301 may be precisely identified, the start of progress of each stage may be precisely controlled, and the end of the operation may be precisely controlled as necessary.

As a result, each stage of the stapling process may be precisely controlled. For example, the start of each stage may be appropriately controlled. As a specific example, after the working member 540 moves forward in a state of being in a position adjacent to the proximal portion (a standby state) and the grip state of the first jaw 311 and the second jaw 312 is identified through the sensing member 321, a state (a grip completion state) after blood flows out from tissue between the first jaw 311 and the second jaw 312 may be precisely controlled. Then, the working member 540 may move safely and a stapling state (a firing state) may be performed effectively.

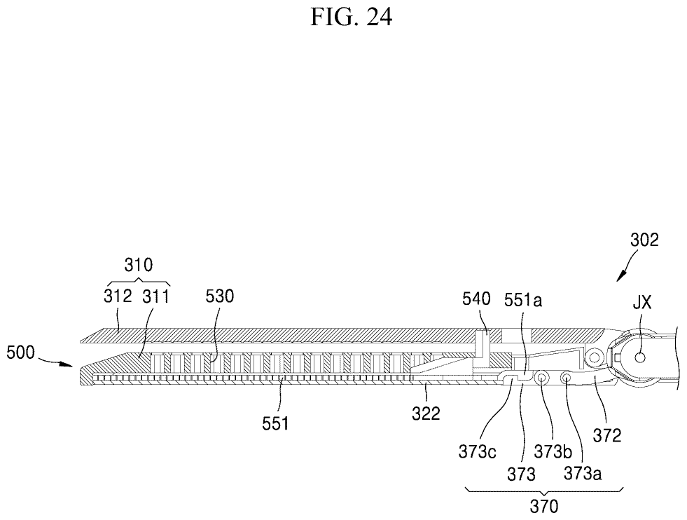

FIG. 24 is a schematic cross-sectional view of an end tool of a surgical instrument according to another embodiment.

Referring to FIG. 24, an end tool 302 according to the present embodiment may include a jaw 310 and a sensing member 322. Since most of the configurations, including the jaw 310, are the same as the embodiment of FIG. 23 or may be modified and applied within a similar range as necessary, a detailed description thereof is omitted and the differences are mainly described.

The sensing member 322 may be disposed in the jaw 310. As an example, the sensing member 322 may be disposed in one area of a first jaw 311. As an example, the sensing member 322 may be disposed on the bottom surface of the first jaw 311 or between the bottom surface of the first jaw 311 and a cartridge 500.

The sensing member 322 may be configured to identify information about a position of a working member 540. For example, the sensing member 322 may include a position detection sensor. As a specific example, the sensing member 322 may be disposed to overlap the working member 540 on a movement path of the working member 540. As an example, the sensing member 222 may have a shape that is longer than the working member 540 in the longitudinal direction of the first jaw 311 on the movement path.

In an alternative embodiment, the sensing member 322 may include a capacitance-type sensor. In this manner, when the working member 540 moves, The capacitance-type sensor may be used to easily sense the overlap or contact with the working member 540 when the working member 540 moves and to identify the information about the position of the working member 540. In another alternative embodiment, the sensing member 322 may include a pressure sensor. In addition, the sensing member 322 may include various other types of position sensors.

The information about the position of the working member 540 through the sensing member 322 may be transmitted in a form that may be identified by a user.

The information about the position of the working member 540 may be identified through the sensing member 322, the state of each stage when performing operations using the end tool 302 may be precisely identified, the start of progress of each stage may be precisely controlled, and the end of the operation may be precisely controlled as necessary.

For example, a waiting time for blood to flow out from tissue between the first jaw 311 and the second jaw 312 due to the grip of the first jaw 311 and the second jaw 312 may be easily maintained, and a stapling process may be precisely controlled.

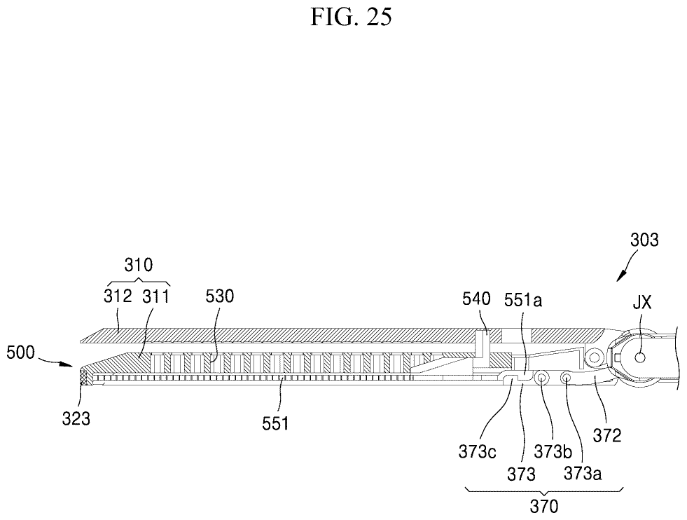

FIG. 25 is a schematic cross-sectional view of an end tool of a surgical instrument according to another embodiment.

Referring to FIG. 25, an end tool 303 according to the present embodiment may include a jaw 310 and a sensing member 323. Since most of the configurations, including the jaw 310, are the same as the embodiment of FIG. 23 or may be modified and applied within a similar range as necessary, a detailed description thereof is omitted and the differences are mainly described.

The sensing member 323 may be disposed in the jaw 310. As an example, the sensing member 323 may be disposed in one area of a first jaw 311. As a specific example, the sensing member 323 may be disposed closer to a distal portion of the first jaw 311. For example, the sensing member 323 may be disposed closer to the distal portion than a position where a cartridge 500 of the first jaw 311 is disposed.

The sensing member 323 may be configured to identify information about a position of a working member 540. For example, the sensing member 323 may include a distance detection sensor.

The distance between the sensing member 323 and the working member 540 may be sensed through the sensing member 323, and the sensed distance or information about the position of the working member 540 through the distance may be transmitted in a form that may be identified by a user.

The information about the position of the working member 540 may be identified through the sensing member 323, the state of each stage when performing operations using the end tool 303 may be precisely identified, the start of progress of each stage may be precisely controlled, and the end of the operation may be precisely controlled as necessary.

For example, a waiting time for blood to flow out from tissue between the first jaw 311 and the second jaw 312 due to the grip of the first jaw 311 and the second jaw 312 may be easily maintained, and a stapling process may be precisely controlled.

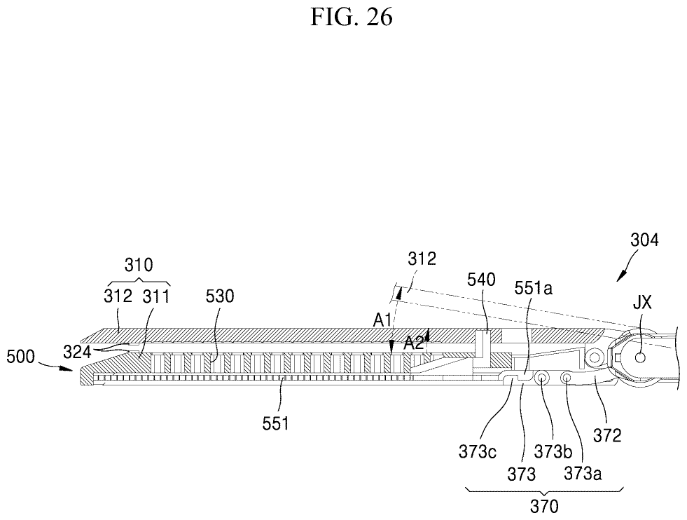

FIG. 26 is a schematic cross-sectional view of an end tool of a surgical instrument according to another embodiment.

Referring to FIG. 26, an end tool 304 according to the present embodiment may include a jaw 310 and a sensing member 324. Since most of the configurations, including the jaw 310, are the same as the embodiment of FIG. 23 or may be modified and applied within a similar range as necessary, a detailed description thereof is omitted and the differences are mainly described.

The sensing member 324 may be configured to sense a value of a gap between a first jaw 311 and a second jaw 312 of the jaw 310, for example, a gap between ends of distal portions or areas adjacent thereto.

As a specific example, the sensing member 324 may be disposed in each of an area adjacent to a distal portion of the first jaw 311 and facing the second jaw 312 and an opposite area adjacent to a distal portion of the second jaw 312 and facing the first jaw 311. In addition, as another example, the sensing member 324 may be disposed in either the first jaw 311 or the second jaw 312.

The sensing member 324 may include a position detection sensor or a distance detection sensor. A gap between the first jaw 311 and the second jaw 312 may be sensed through the sensing member 324, and the sensed distance or information about a position of a working member 540 through the distance may be transmitted in a form that may be identified by a user. For example, a state (a standby state) in which the working member 540 is in a position adjacent to a proximal portion may be identified. This may be transmitted as information that may be identified by the user.

In an alternative embodiment, the end tool 304 may include a sensing member (not shown) that senses an angle A1 between the first jaw 311 and the second jaw 312 and a changed angle A2. In this manner, the gap between the first jaw 311 and the second jaw 312 may be indirectly identified, instead of the sensing members 324 disposed in the first jaw 311 and the second jaw 312. The sensing member that senses the angles A1 and A2 may have various shapes. For example, the sensing member may include an optical angle measuring member and a contact-type angle measuring member. As another example, the sensing member may include a rotation measuring member that measures the amount of rotation when the first jaw 311 and the second jaw 312 rotate around a rotational shaft JX.

The information about the position of the working member 540 may be identified through the sensing member 224 or the sensing member (not shown) that senses the angles A1 and A2, the state of each stage when performing operations using the end tool 304 may be precisely identified, the start of progress of each stage may be precisely controlled, and the end of the operation may be precisely controlled as necessary.

For example, a waiting time for blood to flow out from tissue between the first jaw 311 and the second jaw 312 due to the grip of the first jaw 311 and the second jaw 312 may be easily maintained, and a stapling process may be precisely controlled.

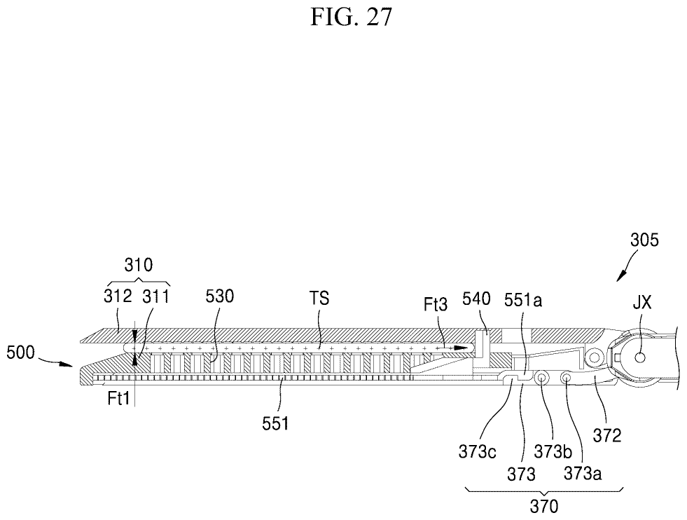

FIG. 27 is a schematic cross-sectional view of an end tool of a surgical instrument according to another embodiment.

Referring to FIG. 27, an end tool 305 according to the present embodiment may include a jaw 310 and a sensing member (not shown). Since most of the configurations, including the jaw 310, are the same as the embodiment of FIG. 23 or may be modified and applied within a similar range as necessary, a detailed description thereof is omitted and the differences are mainly described.

The end tool 305 according to the present embodiment may sense loads received by a first jaw 311 and a second jaw 312 of the jaw 310. For example, when tissue TS is located between the first jaw 311 and the second jaw 312 as illustrated FIG. 21, a load Ft1 received by the first jaw 311 or the second jaw 312 may be sensed.

For example, a pressure sensor and the like may be disposed in one area of the first jaw 311 and the second jaw 312. In addition, a rotation resistance measuring unit that measures resistance in the rotational direction of the first jaw 311 and the second jaw 312 in an area where the first jaw 311 and the second jaw 312 are connected to each other, as a specific example, in an area adjacent to a rotational shaft JX.

In an alternative embodiment, a working member 540 may include a sensing member that senses a load Ft3 the working member 540 receives from the tissue TS when the tissue TS is disposed between the first jaw 311 and the second jaw 312.

On the other hand, in an alternative embodiment, a working guide portion (not shown, sec 240 of FIG. 21) may be further disposed to close the first jaw 311 and the second jaw 312. For example, the working guide portion may have at least two members to support the first jaw 311 and the second jaw 312 on both sides. The load received by at least one of the first jaw 311 and the second jaw 312 through the two members on both sides, for example, the load received in a direction away from each other may be measured.

Through the measurement of the load between the first jaw 311 and the second jaw 312, it may be identified that a state in which the tissue TS is disposed between the first jaw 311 and the second jaw 312, that is, a state (a standby state) in which the working member 540 is in a position adjacent to the proximal portion is maintained without sufficiently closing the first jaw 311 and the second jaw 312. This may be transmitted as information that is recognizable by the user.

In addition, when the working member 540 moves in the direction of a distal portion and the first jaw 311 and the second jaw 312 are closed with the tissue TS therebetween, and thus, the load Ft1 therebetween increases, a change in load may be sensed through the sensing member. In this manner, for example, it may be identified that the working member 540 is moving forward and the first jaw 311 and the second jaw 312 are in a grip state.

After identifying the grip state and passing a grip completion state through a stable standby, the working member 540 may further move forward to an area close to the distal portion. Accordingly, stapling may be performed stably.

As a result, the state of each stage when performing operations using the end tool 305 may be precisely identified, the start of progress of the each stage may be precisely controlled, and the end of the operation may be precisely controlled as necessary.

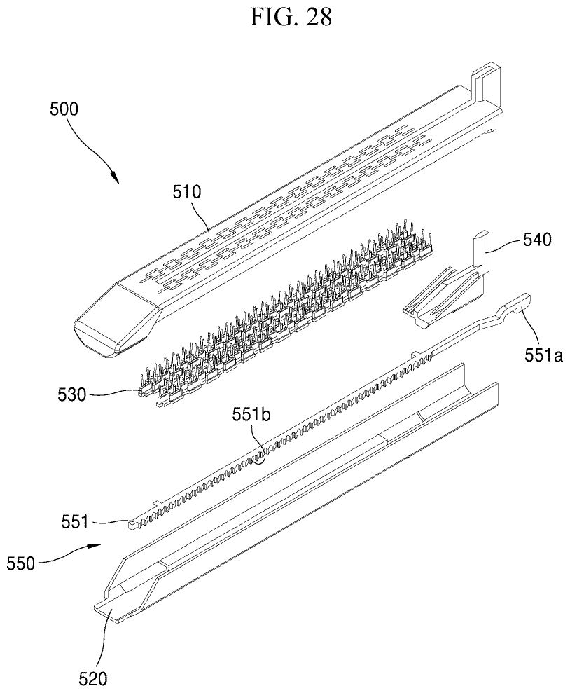

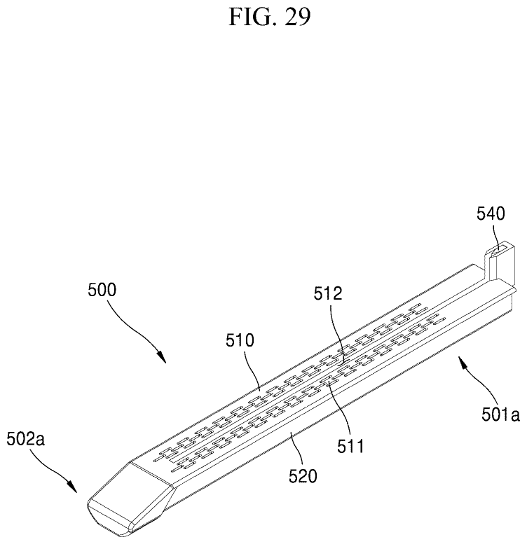

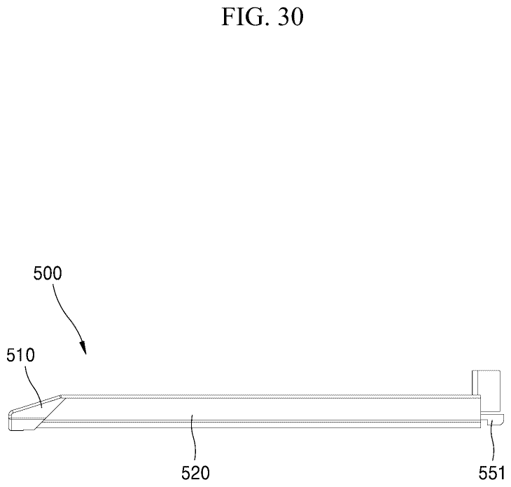

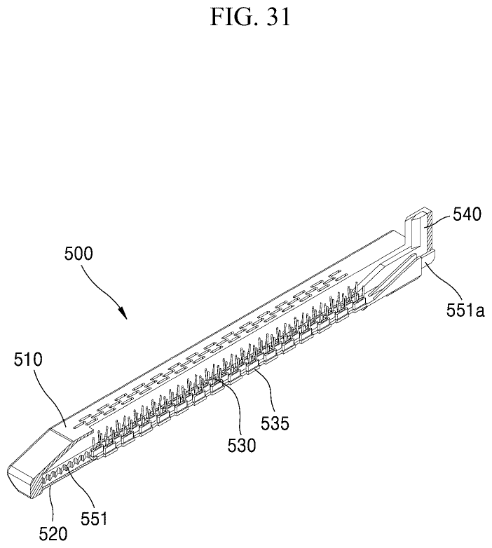

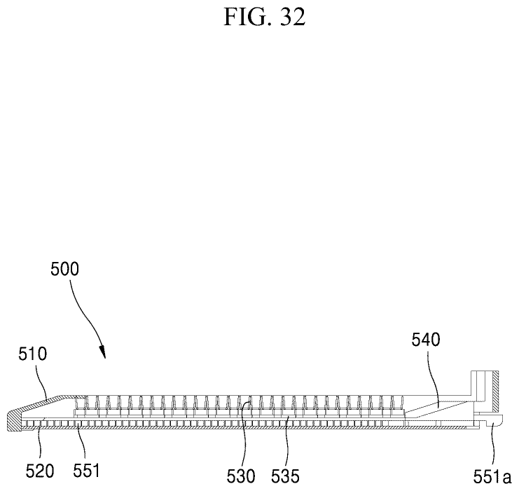

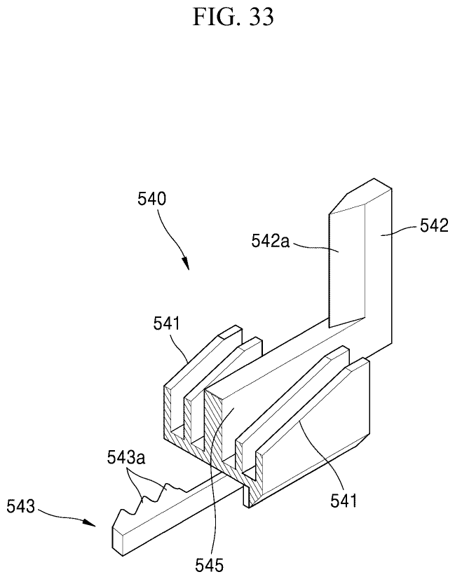

FIG. 28 is an exploded perspective view illustrating a cartridge for an end tool of a surgical instrument according to an embodiment, FIG. 29 is a combined perspective view illustrating the cartridge of FIG. 28, FIG. 30 is a side view illustrating the cartridge of FIG. 28, FIG. 31 is a perspective cross-sectional view illustrating the cartridge of FIG. 28, FIG. 32 is a side cross-sectional view illustrating the cartridge of FIG. 28, and FIG. 33 is a perspective view illustrating a working member in the cartridge of FIG. 28.

For example, the cartridge 500 may be one of the cartridges 500 for the end tools 301 to 305 illustrated in FIGS. 23 to 27. In addition, the cartridge 500 may be optionally applied to the end tools 101 to 103 and 201 to 205 according to the above-described embodiments.

The cartridge 500 may be configured to be attachable to and detachable from a first jaw 311. The cartridge 500 may include a plurality of staples 530 and a blade 542 to perform suturing and cutting of tissue. The cartridge 500 may include a cover 510, a housing 520, a staple 530, a release member 535, a working member 540, and a reciprocating assembly 550.

In addition, in an alternative embodiment, the working member 540 may not be included in the cartridge 500 but may be provided separately from the cartridge 500. For example, the working member 540 may be disposed in the first jaw 311 of a jaw 310. For convenience of explanation, a case where the working member 540 is included in the cartridge 500 is described as an example.

The housing 520 may form the outer shape of the cartridge 500 and may be formed in a shape of a generally hollow box with one surface (an upper surface) removed, so as to accommodate the reciprocating assembly 550, the working member 540, and the staple 530 therein. The housing 520 may have a cross-section of approximately a ‘U’ shape.

The cover 510 may be configured to cover the upper portion of the housing 520. Staple holes 511 through which the staples 530 are ejected to the outside may be formed in the cover 510. The staples 530, which are accommodated in the housing 520 before the stapling operation, are pushed up by the working member 540 during the stapling operation, pass through the staple holes 511 of the cover 510, and are released from the cartridge 500 to perform stapling.

On the other hand, a slit 512 may be formed in the cover 510 in a length direction. The blade 542 of the working member 540 may protrude outward from the cartridge 500 through the slit 512. As the blade 542 of the working member 540 passes along the slit 512, the stapled tissue may be cut.

A plurality of staples 530 may be disposed in the housing 520. As the working member 540 moves linearly in one direction, the staples 530 may be sequentially pushed up from the inside to the outside of the housing 520, thereby performing suturing, that is, stapling. A material of the staples 530 may include titanium, stainless steel, etc.

On the other hand, the release member 535 may be further disposed between the housing 520 and the staple 530. In other words, it may be stated that the staple 530 is disposed on the upper portion of the release member 535. In this case, the working member 540 may push up the release member 535 while moving linearly in one direction, and the release member 535 may push up the staples 530.

This may include a case where the working member 540 directly pushes up the staples 530 and a case where the working member 540 pushes up the release member 535 and the release member 535 pushes up the staples 530 (that is, a case where the working member 540 indirectly pushes up the staples 530). It may be stated that the working member 540 pushes up the staples 530.

The reciprocating assembly 550 may be disposed below the inside of the housing 520. The reciprocating assembly 550 may include one or more reciprocating members 551. In the present embodiment, one reciprocating member 551 is illustrated, but in an alternative embodiment, a plurality of reciprocating members 551 may be provided.

In the present embodiment, the reciprocating member 551 may be a rack. The reciprocating member 551 may include an uneven portion 551b and a fastening portion 551a. In detail, the reciprocating member 551 may be formed in an elongated bar shape, and a plurality of uneven portions 551b having a sawtooth shape may be formed on one surface of the reciprocating member 551. The uneven portion 551b may be formed to be in contact with the working member 540, particularly a ratchet member 543 of the working member 540. In other words, the reciprocating member 551 may include a plurality of uneven portions 551b that engage with the ratchets 543a of the ratchet member 543.

On the other hand, although not illustrated, the reciprocating member 551 may be provided with, in addition to the rack form, a member with various shapes that is directly or indirectly connected to a power transmission member (e.g., a pulley) and performs a linear reciprocating motion according to the rotational motion of the power transmission member (e.g., the pulley). For example, the reciprocating member 551 may be in the form of a clutch having no uneven portion.

The reciprocating member 551 may not be fixedly coupled to other elements of the cartridge 500 and may be configured to be relatively movable with respect to other elements of the cartridge 500. That is, the reciprocating member 551 may perform a linear reciprocating motion with respect to the housing 520 and the cover 510 coupled to the housing 520.

On the other hand, the fastening portion 551a may be formed on a proximal portion 501a of the reciprocating member 551, and the fastening portion 551a may be coupled to the staple link assembly (see 370 of FIG. 23). Therefore, when the staple link assembly 370 performs a linear reciprocating motion, the reciprocating member 551 coupled thereto may also perform a linear reciprocating motion in the same direction.

The working member 540 may be disposed in the housing 520. The working member 540 may be formed to be in contact with the reciprocating member 551 and may be configured to move linearly in one direction according to the linear reciprocating motion of the reciprocating member 551. In other words, the working member 540 may interact with the reciprocating member 551 to perform stapling and cutting while moving in one direction, for example, in a direction from the proximal portion to the distal portion of the end tool (see 301 of FIG. 23).

The working member 540 may include a wedge 541, a blade 542, a ratchet member 543, and a main body 545.

The main body 545 may be formed in a long rectangular pillar shape and may form a base portion of the working member 540.

The wedge 541 may be formed on at least one side of the main body 545 and may be formed to have a certain inclined surface. That is, the wedge 541 may be formed to be inclined to a certain degree with respect to the forward direction of the working member 540. In other words, the cartridge 500 may be formed so that the proximal portion 501a is higher than a distal portion 502a. In the drawings, it is illustrated that two wedges 541 are formed on each of left and right sides of the main body 545, but the disclosure is not limited thereto. The wedge 541 may be formed in various numbers and shapes depending on the shape of the staples 530 or the release member 535 that is in contact with the wedge 541.

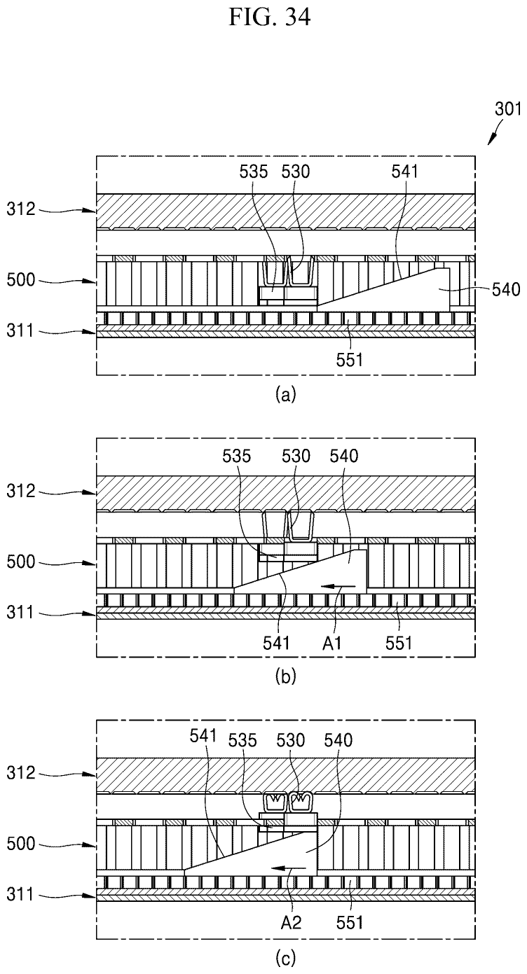

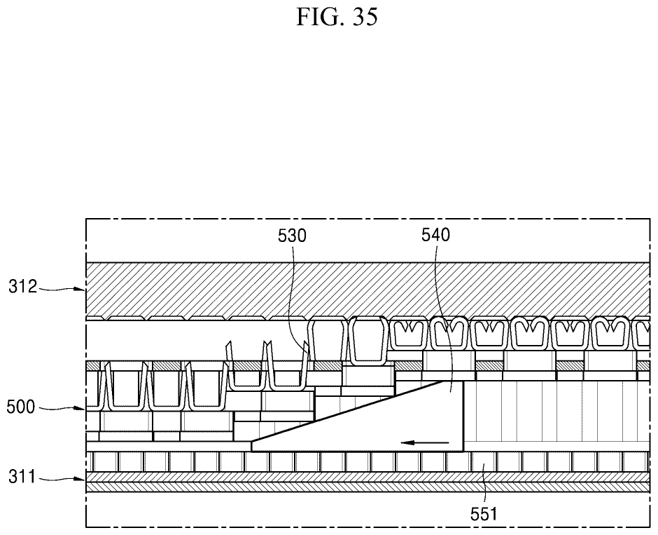

The wedge 541 may be formed to be in sequential contact with the release member 535 or the staples 530 and may serve to push up the staples 530 in turn. As illustrated in FIGS. 34 and 35 to be described below, while the working member 540 moves toward the distal portion 502a, the staples 530 may be sequentially pushed up and released from the cartridge 500.

The blade 542 may be formed on one side of the wedge 541, more specifically, on the side close to the proximal portion 501a of the wedge 541. In one area of the blade 542, a sharp edge portion 542a may be configured to cut tissue. At least a portion of this edge portion 542a may be released from the first jaw (see 311 of FIG. 23) and the cartridge 500 to cut tissue disposed between the first jaw (see 311 of FIG. 23) and the second jaw (see 312 of FIG. 23). The edge portion 542a of the blade 542 may always be released from the first jaw (see 311 of FIG. 23). Alternatively, the edge portion 542a of the blade 542 may be normally accommodated in the first jaw (see 311 of FIG. 23) or the cartridge 500, and only when the working member 540 moves in the longitudinal direction, the edge portion 542a may be released from the first jaw (see 311 of FIG. 23).

The ratchet member 543 may be formed on one side of the wedge 541, more specifically, at the lower portion of the wedge 541, and may be formed to face the reciprocating member 551. The ratchet member 543 may be formed in a bar shape and may include a plurality of ratchets 543a on one surface. Due to the ratchet member 543, the working member 540 may move in only one direction (i.e., toward the distal portion of the cartridge) with respect to the reciprocating member 551. The ratchet 543a of the ratchet member 543 may be formed to be in contact with the uneven portion 551b of the reciprocating member 551.

In an alternative embodiment, an elastic member (not shown) may be formed on one side of the main body 545 or the wedge 541 to apply certain elastic force to the ratchet member 543. As an example, one area of the elastic member may be connected to the wedge 541 or the main body 545, another area of the elastic member may be connected to the ratchet member 543, and the elastic member (not shown) may connect the wedge 541 or the main body 545 to the ratchet member 543. The elastic member may apply elastic force in a direction in which the ratchet member 543 is in close contact with the reciprocating member 551.

The ratchet 543a of the ratchet member 543 may be formed so that a first surface (specifically, a surface of the distal portion 502a) has a certain angle and a gentle slope, and a second surface (specifically, a surface of the proximal portion 501a) is vertical or close to vertical.

In order to engage with the ratchet 543a of the ratchet member 543, the uneven portion 551b of the reciprocating member 551 may be formed so that a first surface (specifically, a surface of the proximal portion 501a) has a certain angle and a gentle slope, and a second surface (specifically, a surface of the distal portion 502a) is vertical or close to vertical.

With this configuration, the ratchet 543a and the uneven portion 551b may act as a type of ratchet in a state of being fastened (or engaged) with each other and may move in only one direction.

In another aspect, when the reciprocating member 551 moves in the direction of the distal portion 502a while the reciprocating member 551 and the ratchet member 543 are fastened (or engaged or in close contact) with each other, the ratchet member 543 may move together toward the distal portion 502a by the reciprocating member 551.

In contrast, when the reciprocating member 551 moves in the direction of the proximal portion 501a while the reciprocating member 551 and the ratchet member 543 are fastened (or engaged or in close contact) with each other, only the reciprocating member 551 may move toward the proximal portion 501a while the ratchet member 543 is in a fixed state.

FIGS. 34 and 35 are diagrams for describing the stapling operation of the end tool to which the cartridge of FIG. 28 is applied.

As a specific example, FIG. 34 is a diagram illustrating each section of the stapling operation of one of the end tools of FIGS. 23 to 27 to which the cartridge of FIG. 28 is applied, and FIG. 35 is a diagram illustrating the overall stapling operation of the end tool to which the cartridge is applied.

Referring to FIGS. 34 and 35, in the state illustrated in (a) of FIG. 34, as the working member 540 moves in the direction of arrow A1 in (b) of FIG. 34, the wedge 541 of the working member 540 pushes up the release member 535, and the release member 535 pushes up the lower side of the staple 530. The staple 530 is ejected from the first jaw 311 and the cartridge 500.

In this state, when the working member 540 moves further in the direction of arrow A2 in (c) of FIG. 34, the ejected staple 530 is continuously pushed up by the working member 540 while being in contact with the anvil of the second jaw 312, both end portions of the staple 530 are bent, and stapling is performed.

As such an operation is continuously performed, stapling is sequentially performed from the staple 530 on the proximal portion side to the staple 530 on the distal portion side among the staples 530, as illustrated in FIG. 35.

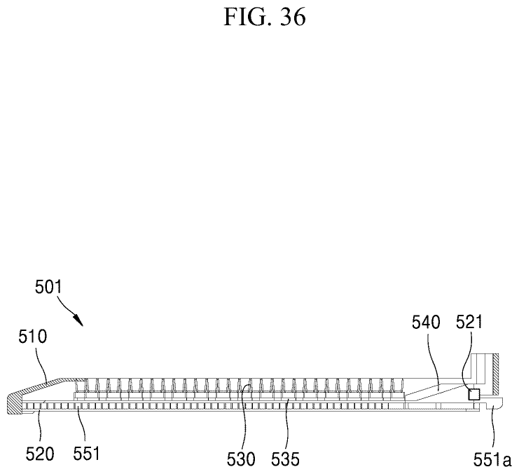

FIG. 36 is a cross-sectional view illustrating a cartridge for an end tool of a surgical instrument according to an embodiment.

A cartridge 501 according to the present embodiment may include a sensing member 521. For example, the cartridge 501 according to the present embodiment may include the sensing member 521 further disposed in the cartridge 500 of FIG. 28. The sensing member 521 may be configured to identify information about a position of a working member 540, and a detailed description thereof may be the same as described in the embodiment of FIGS. 10 to 12.

In the present embodiment, the distance to the working member 540 may be identified through the sensing member 521 disposed in the cartridge 501, and the information about the position of the working member 540 may be identified through the sensing member 221.

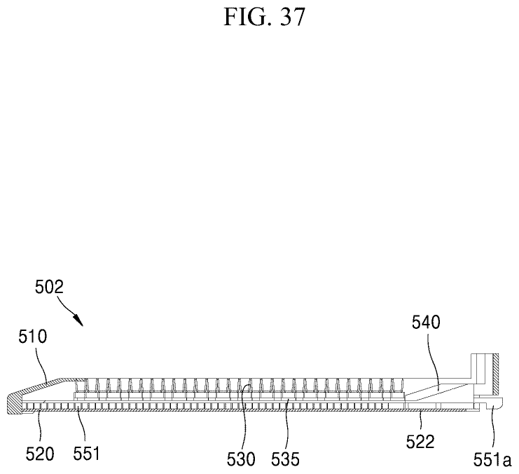

FIG. 37 is a cross-sectional view illustrating a cartridge for an end tool of a surgical instrument according to another embodiment.

A cartridge 502 according to the present embodiment may include a sensing member 522. For example, the cartridge 502 according to the present embodiment may include the sensing member 522 further disposed in the cartridge 500 of FIG. 28. The sensing member 522 may be disposed to overlap the working member 540. For example, the sensing member 522 may be disposed between a bottom surface of a housing 520 and a staple 530.

In the present embodiment, the sensing member 522 disposed in the cartridge 502 may identify the distance to the working member 540 and the information about the position of the working member 540.

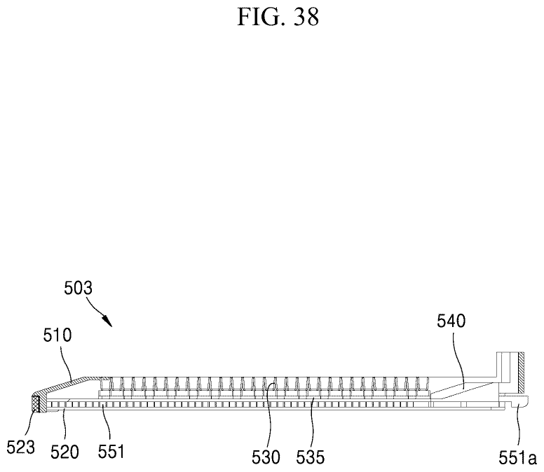

FIG. 38 is a cross-sectional view illustrating a cartridge for an end tool of a surgical instrument according to another embodiment.

A cartridge 503 according to the present embodiment may include a sensing member 523. For example, the cartridge 503 according to the present embodiment may include the sensing member 523 further disposed in the cartridge 500 of FIG. 28. The sensing member 523 may be disposed in one area of a housing 520 and may be disposed close to a distal portion. For example, the sensing member 523 may be disposed closer to the distal portion than a staple 530.

In the present embodiment, the sensing member 522 disposed in the cartridge 503 may identify the distance to the working member 540 and the information about the position of the working member 540.

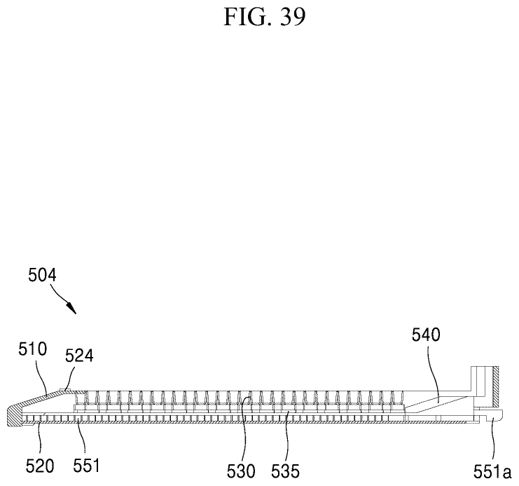

FIG. 39 is a cross-sectional view illustrating a cartridge for an end tool of a surgical instrument according to another embodiment.

A cartridge 504 according to the present embodiment may include a sensing member 524. For example, the cartridge 504 according to the present embodiment may include the sensing member 524 further disposed in the cartridge 500 of FIG. 28. The sensing member 524 may be disposed in one area of a housing 520 or a cover 510. The sensing member 524 may be configured to sense a value of a gap with the second jaw (see 312 of FIG. 23). Accordingly, the sensing member 524 may sense a gap between the first jaw (see 311 of FIG. 23) and the second jaw (see 312 of FIG. 23).

When performing operations using the end tools using the cartridges 501 to 504 of FIGS. 36 to 39, the position of the working member may be easily identified through the sensing member of the cartridge even when there is no sensing member in the end tool. When performing operations using the end tools using the cartridges 501 to 504, the state of each stage may be precisely identified, the start of progress of each stage may be precisely controlled, and the end of the operation may be precisely controlled as necessary.

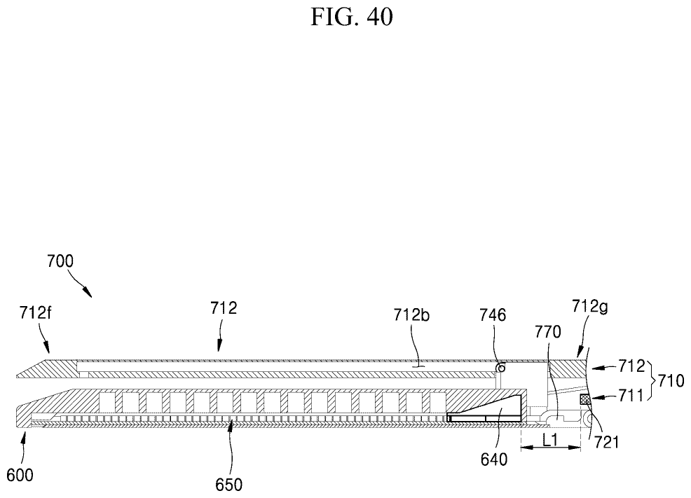

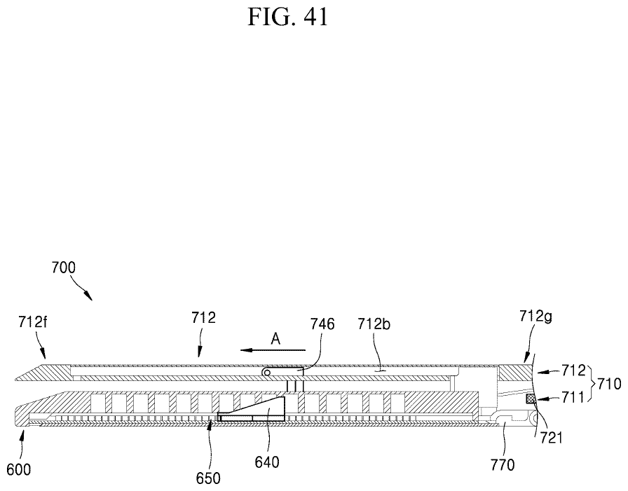

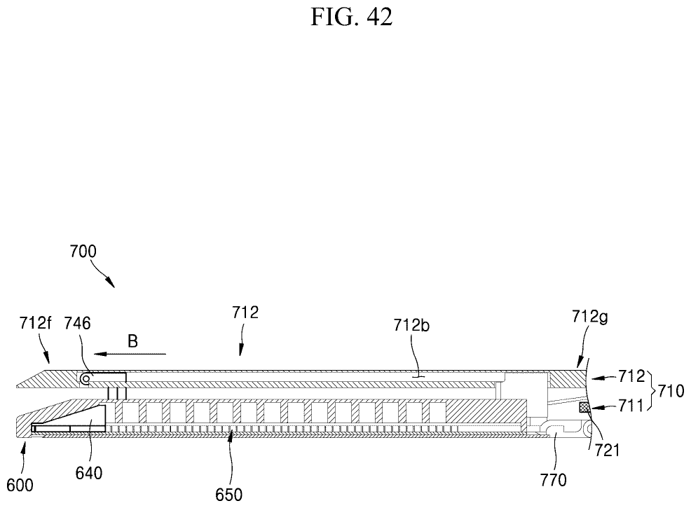

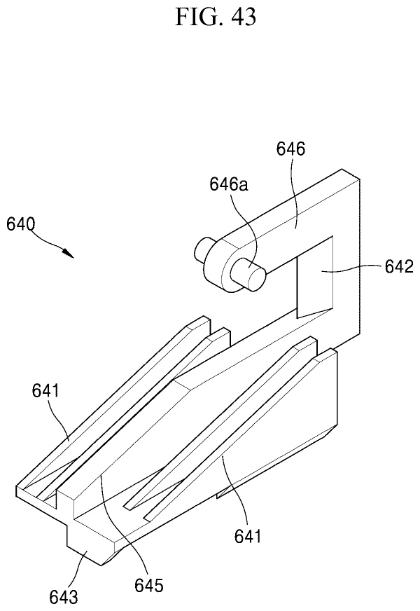

FIG. 40 is a schematic cross-sectional view of an end tool of a surgical instrument according to another embodiment. FIGS. 41 and 42 are diagrams for describing a motion of a working member in the end tool of FIG. 40. FIG. 43 is a perspective view for describing a working member of a cartridge in the end tool of FIG. 40.

Referring to FIGS. 40 to 42, an end tool 700 of a surgical instrument according to the present embodiment may include a jaw 710 and a sensing member 721.

In addition, although not illustrated, an end tool 700 according to the present embodiment may optionally apply various sensing members included in the end tools 301, 302, 303, 304, and 305 in the embodiments of FIGS. 24 to 27.

For convenience of explanation, the end tool 700 according to the present embodiment is described as being applied within substantially the same range as the sensing member 321 included in the end tool 301 of FIG. 24.

A sensing member 721 may be disposed in a jaw 710. As an example, the sensing member 721 may be disposed in one area of a first jaw 711. As a specific example, the sensing member 721 may be disposed close to a proximal portion of the first jaw 711 and may be disposed closer to a proximal portion than a working member 640.

The first jaw 711 may be formed in an elongated bar shape as a whole. The first jaw 711 may be configured to accommodate a cartridge 600 on a distal portion side. A guide groove (not shown) may be formed on one side of the first jaw 711, for example, the proximal portion side, to guide a movement of a staple link assembly 770. A detailed description thereof may be the same as described in the embodiment of FIG. 3 or may be modified and applied within a similar range.

The second jaw 712 may include an anvil (not shown) and a guide groove 712b and may include one or more coupling grooves.

The second jaw 712 may be formed in an elongated bar shape as a whole. An anvil may be formed in an area facing the first jaw 711. A power transmission member (for example, a pulley) may be coupled to a proximal portion 712g so as to be rotatable around a rotational shaft.

The guide groove 712b may be formed inside the second jaw 712 to guide the movement of the working member 640 of the cartridge 600. The guide groove 712b may be formed in a groove shape along a movement path of the working member 640. While a clamp 646 of the working member 640 formed in a protrusion shape is fitted into the guide groove 712b having a groove shape, the clamp 646 may move along the guide groove 712b, and thus, the working member 640 may move relative to the second jaw 712. As a specific example, the working member 640 may move along the guide groove 712b of the second jaw 712.

One or more coupling grooves for the entrance and exit of one or more clamps 646 may be formed in the second jaw 712. Specifically, one or more coupling grooves may be formed in the proximal portion 712g and one or more coupling grooves may be formed in a distal portion 712f. For example, coupling grooves may be formed at both end portions of the guide groove 712b. The clamp 646 of the working member 640 may be inserted into or released from the second jaw 712 through the coupling groove formed in the guide groove 712b.

The cartridge 600 may include a cover (see 510 of FIG. 28), a housing (see 520 of FIG. 28), a staple (see 530 of FIG. 28), a working member 640, and a reciprocating assembly 650. Since other elements of the cartridge 600, except for the working member 640, may be substantially the same as the cartridge (see 500 of FIG. 28) according to the above-described embodiment or may be modified and applied within a similar range, a detailed description thereof is omitted.

The working member 640 may further include a wedge 641, a blade 642, a ratchet member 643, an elastic member (not shown), a main body 645, and a clamp 646.

The clamp 646 may be formed on one side of the blade 642 and may be formed in a shape substantially parallel to the main body 645 or the wedge 641. A protrusion 646a may be formed at one end portion of the clamp 646. The protrusion 646a may move along the guide groove 712b.

In an alternative embodiment, the clamp 646 may be formed parallel to the wedge 641.

It is possible to better withstand strong pressure applied to the wedge 641 during the stapling operation through the movement of the working member 640.

In addition, the closed state of the first jaw 711 and the second jaw 712 and the gap therebetween may be controlled through the working member 640 as desired. In this manner, the thickness of tissue disposed between the first jaw 711 and the second jaw 712 may be compressed to easily ensure stable performance of stapling and incision.

For example, the compression force of the tissue and the uniformity of compression may be improved by applying higher compression force through the working member 640 than applying compression force to the desired tissue between the first jaw 711 and the second jaw 712 only with the grip force of the first jaw 711 and the second jaw 712.

In addition, the widening may be reduced or prevented due to tissue elasticity or the like by efficiently controlling the gap between the first jaw 711 and the second jaw 712 near the working member 640. Accordingly, compressing the tissue to the intended thickness may be performed efficiently.

On the other hand, since the clamp 646 of the working member 640 is accommodated and coupled only to the inside of the second jaw 712, precise stapling control may be implemented by reducing an occurrence of tilt moment in the forward and backward direction that occurs due to frictional force generated on both sides of the first jaw 711 and the second jaw 712 when the working member 640 moves forward.

In order to increase such an effect, the working member 640 may be variously designed according to the shapes of the first jaw 711 and the second jaw 712. As an example, an area that is in contact with the second jaw 712 may include a curved area.

When the first jaw 711 and the second jaw 712 are in a closed state, the clamp 646 may pass through the coupling groove and may be located in the guide groove 712b, as illustrated in FIG. 40. At this time, the position of the working member 640 may be identified by identifying a distance L1 to the working member 640 through the sensing member 721. For example, it may be confirmed that the working member 640 is in a standby state. As a specific example, it may be precisely determined that the working member 640 moves forward and the first jaw 711 and the second jaw 712 move to a closed state (a grip state).

After identifying the grip state, the state (the grip completion state) after blood flows out from tissue disposed between the first jaw 711 and the second jaw 712 may be precisely controlled. Then, as illustrated in FIG. 41, when the working member 640 moves in the direction of arrow A, the clamp 646 may also move along the guide groove 712b. Even in this state, the sensing member 721 may optionally sense the distance to the working member 640.

In this state, as illustrated in FIG. 42, when the working member 640 continues to move along the direction of arrow B and enters a stapled state (firing state) with respect to the tissue and the working member 640 reaches the distal portion 712f of the second jaw 712, the clamp 646 may pass through the coupling groove and may be released from the guide groove 712b.

Accordingly, stapling may be performed stably and efficiently through precise control.

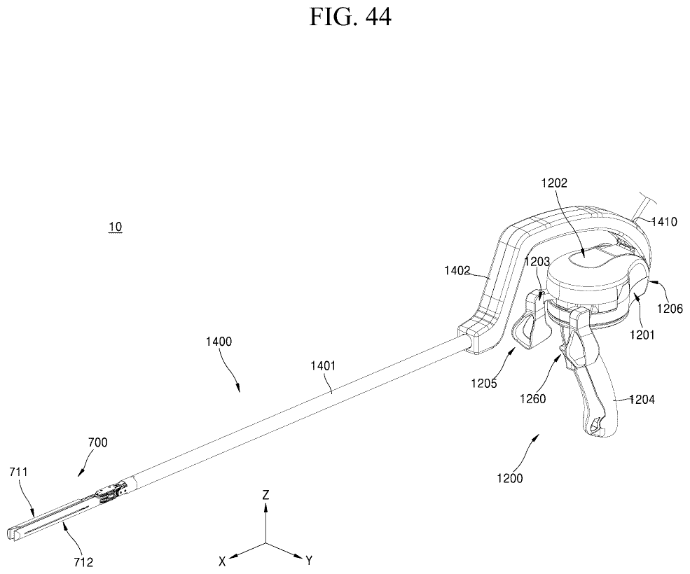

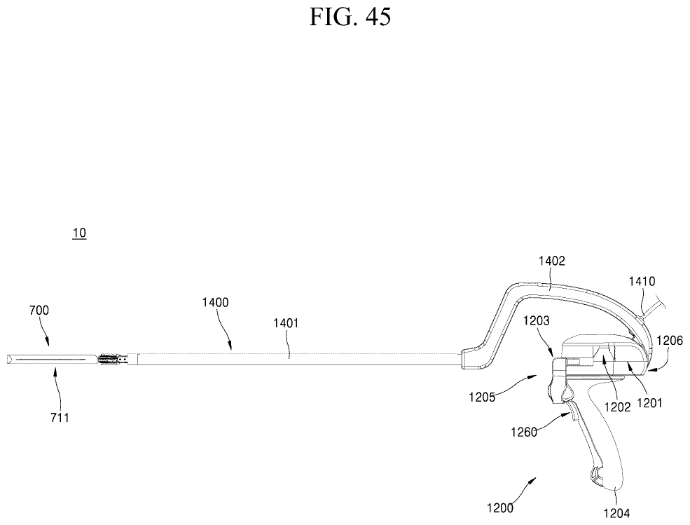

FIG. 44 is a schematic perspective view of a surgical instrument according to an embodiment and FIG. 45 is a side view of the surgical instrument of FIG. 44.

Referring to FIGS. 44 and 43, a surgical instrument 10 according to the present embodiment may include an end tool 700, a manipulation portion 1200, a power transmission unit (not shown), and a connection portion 1400.

Since the end tool 700 may include the end tool 700 of the embodiment illustrated in FIG. 40 as it is, a detailed description thereof is omitted.

In addition, although not illustrated, in an alternative embodiment, the surgical instrument 10 may optionally apply one of the end tools described above.

The connection portion 1400 may be formed in a hollow shaft shape so that one or more wires and electric wires may be accommodated therein. The manipulation portion 1200 may be coupled to one end of the connection portion 1400, and the end tool 700 may be coupled to the other end of the connection portion 1400. Accordingly, the connection portion 1400 may serve to connect the manipulation portion 1200 to the end tool 700.

In an alternative embodiment, the connection portion 1400 may include a straight portion 1401 and a bent portion 1402. The straight portion 1401 may be formed on the side to which the end tool 700 is coupled and the bent portion 1402 may be formed on the side to which the manipulation portion 1200 is coupled. As such, since the end of the connection portion 1400, which is close to the manipulation portion 1200, is bent, a pitch manipulation portion 1201, a yaw manipulation portion 1202, and an actuation manipulation portion 1203 may be formed on an extension line of the end tool 700 or may be formed adjacent to the extension line. In another aspect, it may be stated that at least a portion of each of the pitch manipulation portion 1201 and the yaw manipulation portion 1202 is accommodated in a concave portion formed by the bent portion 1402. Due to the shape of the bent portion 1402, the shapes and operations of the manipulation portion 1200 and the end tool 700 may be consistent more intuitively.

On the other hand, a plane in which the bent portion 1402 is formed may be a pitch plane, that is, substantially the same plane as an XZ plane of FIG. 44. As such, since the bent portion 1402 is formed on substantially the same plane as the XZ plane, interference between the manipulation portions be reduced. For intuitive operations of the end tool and the manipulation portion, configurations other than the XZ plane may also be possible.

On the other hand, in an alternative embodiment, a connector 1410 may be formed in the bent portion 1402. The connector 1410 may be connected to an external power source (not shown). In addition, the connector 1410 may be connected to the end tool 700 through an electric wire. Accordingly, electrical energy supplied from the external power source (not shown) may be transmitted to the end tool 700.

The manipulation portion 1200 may be formed at one end of the connection portion 1400 and may be provided with an interface that may be directly manipulated by a doctor, for example, a handle 1204, a tweezers shape, a stick shape, or a lever shape. When the doctor manipulates this, the end tool 700 that is connected to the interface and inserted into a body of a surgical patient performs a certain operation so that surgery is performed. Although it is illustrated that the manipulation portion 1200 is formed in a handle shape that is rotatable with a finger inserted thereinto, the disclosure is not limited thereto. Various types of manipulation portions connected to the end tool 700 to manipulate the end tool 700 are possible. In addition, various controls on the manipulation portion 1200 may be possible through one or more driving shafts disposed in a direction from the distal portion 1205 to the proximal portion 1206 of the manipulation portion 1200.

In addition, the manipulation portion 1200 of the surgical instrument 10 may further include a staple manipulation portion 1260 that performs stapling and cutting of the end tool 700.

On the other hand, the present embodiment may also be applied to a robot-driven type, instead of a manual type that is directly controlled by a person. In this case, the end tool 700 may be connected directly to a robot or may be connected to a robot driving arm or other areas through the connection portion 1400, instead of the manual-type manipulation portion 1200.

The end tool 700 of the surgical instrument 10 according to the present embodiment may be configured to be rotatable in at least one direction. For example, the end tool 700 of the surgical instrument 10 according to the present embodiment may be configured to perform a pitch motion around the Y-axis of FIG. 44 and simultaneously perform a yaw motion and an actuation motion around the Z-axis.

The pitch, yaw, and actuation motions as used herein are defined as follows.

The pitch motion refers to a motion in which the end tool 700 rotates in the up/down direction with respect to the extension direction (X-axis direction in FIG. 44) of the connection portion 1400, that is, a motion in which the end tool 700 rotates around the Y-axis in FIG. 44. In other words, the pitch motion refers to a motion in which the end tool 700 extending from the connection portion 1400 in the extension direction (X-axis direction in FIG. 44) of the connection portion 1400 rotates up and down around the Y-axis with respect to the connection portion 1400.

The yaw motion refers to a motion in which the end tool 700 rotates in the left/right direction with respect to the extension direction (X-axis direction in FIG. 44) of the connection portion 1400, that is, a motion in which the end tool 700 rotates around the Z-axis in FIG. 44. In other words, the yaw motion refers to a motion in which the end tool 700 extending from the connection portion 1400 in the extension direction (X-axis direction in FIG. 44) of the connection portion 1400 rotates left and right around the Z-axis with respect to the connection portion 1400. In other words, the yaw motion refers to a motion in which the two jaws 711 and 712 formed in the end tool 700 rotate in the same direction around the Z-axis.

On the other hand, the actuation motion refers to a motion in which the end tool 700 rotates around the same rotational shafts as the yaw motion and the two jaws 711 and 712 are retracted or spread while rotating in opposite directions. In other words, the actuation motion refers to a motion in which the two jaws 711 and 712 formed in the end tool 700 rotate in opposite directions around the Z-axis.

The power transmission unit (not shown) may connect the manipulation portion 1200 to the end tool 700 and serve to transmit the driving force of the manipulation portion 1200 to the end tool 700. The power transmission unit may include a plurality of wires, pulleys, links, nodes, gears, etc.

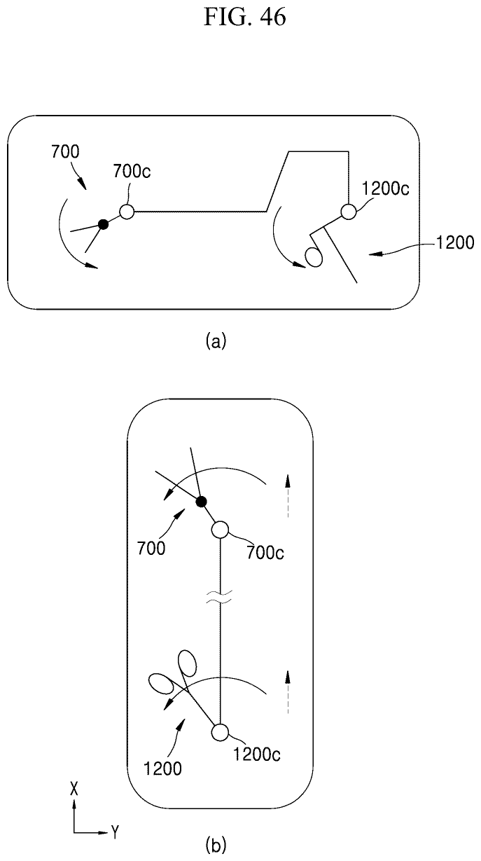

FIG. 46 is a diagram for schematically describing an operation concept of a surgical instrument according to an embodiment.

For example, an example of the operation of the surgical instrument 10 of FIGS. 45 and 46 is described with reference to FIG. 46.

Specifically, referring to (a) and (b) of FIG. 46, in the surgical instrument, the end tool 700 is formed in front of a rotation center 700c of the end tool, and the manipulation portion 1200 is also formed in front of a rotation center 1200c of the manipulation portion. Accordingly, the operations of the manipulation portion 1200 and the end tool 700 are intuitively consistent. To express these characteristics differently, the surgical instrument according to an embodiment of the disclosure is formed so that at least a portion of the manipulation portion may be closer to the end tool (than its own joint) with respect to its own joint at more than one point in the manipulation process.

Since both the end tool and the manipulation portion of the surgical instrument according to an embodiment of the disclosure move based on the rotation center formed at the rear, it may be stated that operations are intuitively consistent in terms of structure. To explain it differently, just as the moving portion of the end tool moves based on the rotation center formed at the rear, the moving portion of the manipulation portion also moves based on the corresponding rotation center formed at the rear. Accordingly, in terms of structure, it may be stated that the operations are intuitively consistent. This allows users to quickly and intuitively control the direction of the end tool, which reduces the possibility of making mistakes.

In an alternative embodiment, the surgical instrument to which the end tool according to the present embodiment is applied may be driven in various ways. For example, the disclosure may be applied to embodiments in which the central axis of the end tool is changed back and forth, or the manipulation portion and the central axis thereof are changed back and forth.

The disclosure has been described with reference to the embodiments illustrated in the drawings, but this is only an example. It will be understood by those of ordinary skill in the art that various modifications and equivalents may be made thereto. Accordingly, the true technical protection scope of the disclosure should be defined by the technical spirit of the appended claims.

The embodiments may be represented by functional block configurations and various processes. Such functional blocks may be implemented in various numbers of hardware and/or software configurations that perform specific functions. For example, the embodiments may employ integrated circuit configurations, such as memory, processing, logic, look-up table, etc., which are capable of executing various functions under the control of one or more microprocessors or other control devices,

The specific executions described in the embodiments are only embodiments and do not limit the scope of disclosure in any way. For the brevity of the specification, descriptions of conventional electronic components, control methods, software, and other functional aspects of the methods may be omitted. In addition, connecting lines or connecting members illustrated in the drawings are intended to represent exemplary functional connections and/or physical or circuit connections. In an actual device, it may appear as a variety of alternative or additional functional, physical, or circuit connections. In addition, when there is no specific mention such as “essential,” “important,” etc., it may not be a necessary component for the application of the disclosure.

The use of the term “the” and similar demonstratives in the context of describing the present specification (especially in the context of the claims) is to be construed to cover both the singular and the plural. In addition, when a range is described in the embodiments, it includes the invention to which individual values within the range are applied (unless otherwise indicated herein). This is the same as stating each individual value constituting the above range in the detailed description of the disclosure. Finally, operations constituting the methods according to the disclosure may be performed in any suitable order unless otherwise indicated herein or otherwise clearly contradicted by context. The embodiments are not necessarily limited by the order of operations. The use of any and all examples or exemplary terms (e.g., “such as”) provided herein is simply intended to describe the embodiments in detail, and the scope of the embodiments is not limited by the examples or exemplary terms unless otherwise claimed. In addition, it will be understood by those of ordinary skill in the art that various modifications, combinations and changes may be made according to design conditions and factors within the scope of the appended claims or equivalents thereof.

The end tool, the cartridge, the surgical instrument, and the surgical instrument driving method according to the disclosure may facilitate precise control of operation progress when one or more operations included in laparoscopic surgery or various other surgeries are performed.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An end tool of a surgical instrument, the end tool comprising a jaw and a sensing member,
wherein the jaw is configured to accommodate at least one area of a working member that is movable in one direction, and
the sensing member is configured to sense information about a position of the working member.

2. The end tool of claim 1, wherein the sensing member is further configured to sense a distance to the working member.

3. The end tool of claim 2, wherein the sensing member is disposed closer to a proximal portion of the end tool than the working member.

4. The end tool of claim 2, wherein the sensing member is disposed closer to a distal portion of the end tool than the working member.

5. The end tool of claim 1, wherein the sensing member is disposed in an overlapped position when the working member moves and is configured to sense the position of the working member.

6. The end tool of claim 1, wherein the jaw comprises a first jaw and a second jaw facing each other and disposed to move closer to or farther away from each other.

7. The end tool of claim 6, wherein the first jaw and the second jaw of the jaw are configured to rotate around one shaft so as to be closer to or farther away from each other.

8. The end tool of claim 6, wherein the sensing member is further configured to sense a distance between the first jaw and the second jaw.

9. The end tool of claim 6, wherein the sensing member is further configured to sense a load that the first jaw or the second jaw receives from a body tissue when the body tissue is located between the first jaw and the second jaw.

10. The end tool of claim 7, wherein the sensing member is further configured to sense a rotation angle when the first jaw and the second jaw are coupled to each other to perform a rotation motion.

11. The end tool of claim 1, wherein the jaw is further configured so that a staple is disposed thereon, and
the staple moves through movement of the working member to perform stapling.

12. The end tool of claim 11, wherein a wedge portion of the working member pushes up the staple through the movement of the working member, and the stapling is performed while a blade of the working member performs a cutting operation.

13. The end tool of claim 11, wherein the jaw is further configured to accommodate a cartridge, and
the staple is provided in plurality in the cartridge.

14. A cartridge applied to an end tool of a surgical instrument, the cartridge comprising:
a housing;
a plurality of staples disposed in the housing;
a working member configured to be movable in at least one direction; and
a sensing member configured to sense information about a position of the working member.

15. The cartridge of claim 14, wherein the sensing member is further configured to sense a distance to the working member.

16. The cartridge of claim 15, wherein the sensing member is disposed closer to a proximal portion of the cartridge than the working member.

17. The cartridge of claim 15, wherein the sensing member is disposed closer to a distal portion of the cartridge than the working member.

18. The cartridge of claim 14, wherein the sensing member is disposed in an overlapped position when the working member moves and is configured to sense the position of the working member.

19. The cartridge of claim 14, wherein the working member comprises:
a wedge portion having an inclined surface; and
a blade formed on one side of the wedge portion and having an edge portion.

20. A surgical instrument comprising:
the end tool of claim 1;
a manipulation portion configured to control an operation of the end tool; and
a connection portion connecting the manipulation portion to the end tool.
